(19) United States
(12) Patent Application Publication
(10) Pub. No.: US 2025/0263747 A1
(43) Pub. Date: Aug. 21, 2025

(54) Higher yields and improved recombinant adeno-associated virus vectors by altering intracellular trafficking in producer cells.

(71) Applicant: peter ward, New York, NY (US)

(72) Inventor: peter ward, New York, NY (US)

(21) Appl. No.: 19/082,850

(22) Filed: Mar. 18, 2025

(51) Int. Cl. C12N 15/86

(52) U.S. Cl. CPC C12N 15/86; C12N 2750/14143; C12N 2750/14152

(57) ABSTRACT

Chemical agents are added to standard production methods for recombinant adeno-associated viral vector to alter intracellular trafficking pathways in producer cells. This achieves, per producer cell, the synthesis of more genome containing vector particles and particles that are better transducing agents. Vector so produced also has: a greater propensity for export out of the producer cell, a more gradual transduction of infected cells, an enhanced ability for integration into the genome of cells that are coinfected by the adeno-associated virus, and an increased probability of transforming cells with multiple vectors.

P. Ward, N. Clément, R. M. Linden, cis effects in adeno-associated virus type 2 replication. J Virol 81, 9976-9989 (2007).

Xiao, P. J. & Samulski, R. J. Cytoplasmic trafficking, endosomal escape, and perinuclear accumulation of adeno-associated virus type 2 particles are facilitated by microtubule network. J. Virol. 86, 10462-10473 (2012)

M. Nonnenmacher, H. van Bakel, R. J. Hajjar, T. Weber, High capsid-genome correlation facilitates creation of AAV libraries for directed evolution. Mol Ther 23, 675-682 (2015).

L. P. van Lieshout et al., A novel dual-plasmid platform provides scalable transfection yielding improved productivity and packaging across multiple AAV serotypes and genomes. Mol Ther Methods Clin Dev 29, 426-436 (2023).

Rongze Yang, et al. AAVone: A Cost-Effective, Single-Plasmid Solution for Efficient AAV Production with Reduced DNA Impurities. Posted to BioRxiv. January 2025.

V. V. Emmerling et al., Rational plasmid design and bioprocess optimization to enhance recombinant adeno-associated virus (AAV) productivity in mammalian cells. Biotechnol J 11, 290-297 (2016).

D. Grimm, M. A. Kay, J. A. Kleinschmidt, Helper virus-free, optically controllable, and two-plasmid-based production of adeno-associated virus vectors of serotypes 1 to 6. Mol Ther 7, 839-850 (2003).

### FIELD OF THE INVENTION

Production of recombinant adeno-associated virus vectors that are used in gene therapy.

### BACKGROUND OF THE INVENTION

Glossary: There is a definition of phrases used in this application to be found at the beginning of the “Detailed Description” section.

### Brief Summary of AAV and rAAV Biology

AAV refers to the adeno-associated virus. rAAV refers to a recombinant AAV, which is the vector in this application.

Recombinant adeno-associated virus (rAAV) vectors have become a prominent delivery vehicle in gene therapy. The reasons for their prominence: the virus is non-pathogenic, it is non autonomous (i.e. it does not replicate on its own), it is able to infect many human cell types, it is stable and amenable easy storage, it is less immunogenic than many viruses, and the viral capsids can be modified to alter target cell specificity. Genes delivered in rAAV vectors have relatively persistent expression. The most important attributes are non-pathogenicity, since the vectors are infused into human patients, and persistent expression.

AAV is a small, non-enveloped, single stranded DNA virus. At each end of the genome are hairpin structures of 145 bases called the Inverted Terminal Repeats (ITRs). The ITRs contain the origin of replication for the genome as well as the signals that enable packaging of the genome into the viral capsid. When using the virus as a gene therapy vector the entirety of its internal sequences can be, and usually are, removed and replaced with a gene of interest (GOI) and regulatory sequences for this gene. (FIG. 1) The only wild-type AAV (wt AAV) sequences remaining in the rAAV vector genome are the ITRs.

After AAV or rAAV infection the viral particle migrates to the nucleus and is uncoated, whether outside the nucleus or after a viral particle is transported into the nucleus. (“uncoating” is a somewhat vague term; it is used herein to mean the measurable evidence that the viral genome has been released from the nucleocapsid.) In this application it is the GFP (Green Fluorescent Protein) gene and regulatory elements that are encapsidated in the rAAV vector., i.e. they are the GOI. (The GFP gene is a commonly used marker gene for assessing the effectiveness of gene delivery systems). What is measured is green light from the GFP protein when cells are irradiated with UV light. What is required is that the GFP gene be transcribed and translated and processed into functional protein.

This delivery of the rAAV genome to the nucleus is thought to happen rapidly. Only a very small fraction of the rAAV delivered to cells results in a successful expression of the gene of interest. The reasons for this inefficiency are unknown. This inefficiency is not found in the wt AAV. The contrast between the high infection efficiency of the wt AAV and the low transduction efficiency of rAAV implies that present production methods for rAAV produce vector particles that are less functional than wt AAV particles.

After the rAAV genome has been released into the nuclear space, it is rapidly converted into a double stranded form, either by the annealing of DNA strands of opposite polarity or extension by host cell polymerases of the single stranded form. The hairpin structures of the ITRs at the ends of its genome provide an efficient primer for this extension. The vector genomes in the rAAV infected cell remain predominantly episomal though some do integrate into the host cell's genome, apparently at random locations. Integration is more likely if the cells are replicating at the time of infection. Gene expression from these vector genomes, whether episomal or integrated, provides the therapeutic effect.

The production of rAAV vectors is described in the Detailed Description.

Problems with rAAV vectors. What is needed.

This application addresses several problems with rAAV as a vector. Insufficient quantities of rAAV vectors for pre-clinical and clinical trials is currently a bottleneck. This is in part due to the yield of functional vector particles per producer cell being too low, much lower with rAAV vectors than it is with production of the virus after infection by the wild type AAV (wt AAV). In addition, as mentioned above, most of the produced rAAV are apparently non-productive. When a population of cells is infected with rAAV the number of vector particles required to successfully transduce one cell is often as high as 10,000 or more genome containing particles per cell, necessitating the use of large quantities of vector. Gene therapy treatments with rAAV vectors are currently expensive, one reason being the difficulty of producing vector. (Several FDA approved gene therapies using rAAV vectors cost in excess of $ 3,000,000 for one treatment.) A second problem is that while AAV is non immunogenic compared to other viruses the large quantities of vector required for gene therapy does provoke an immune response. There have been several deaths and other adverse events in children and young adults treated with rAAV for Duchenne muscular dystrophy, x-linked myotubular myopathy, and spinal muscular atrophy. Adverse events have corelated with vector dose. The cause of deaths seems most often multi organ failure due to a “cytokine storm”. This is an effect often described with infection by large amounts of virus; the doses of rAAV vector given in these treatments have been as high as 1×10¹⁴ vg/kg body weight (Here vg stands for vector genomes so this is the number of vector particles that contain the rAAV genome, thereby discounting empty particles, which can be substantial in number.) This immunotoxicity seem due to both innate and adaptive responses to the vector. Most humans had an inapparent infection by AAV in childhood or adolescence and retain some level of antibodies to the virus.

Separation of the genes needed for vector production on separate plasmids in the producer cells may contribute to the inefficiency of vector production. There are reports of producing higher levels of vector by consolidating the genes required for vector production onto two or one plasmids, but the improvement, as reported, has been modest. A possible reason for only limited improvement is that while consolidated vectors may overcome the “separation of genes problem”, larger vectors are less readily taken up by the producer cells during transfection.

Vector production methods that give increased yields per producer cell and produce a vector that transduces target cells more efficiently than one transduced cell per 10,000 vector particles would be useful to reduce cost of vector and severity of the immune response.

### SUMMARY OF THE INVENTION

A major effort is being made to develop rAAV vector production methods to give higher yields per producer cell. This application describes a modification to standard rAAV vector production methods to do this. The modification is based on disrupting the communications and trafficking networks within the producer cells.

Inherent in the standard methods for producing rAAV vector is the separation in the producer cell of the genes coding for the components required for vector production due to their delivery on separate plasmids. The synthesis of rAAV vector particles requires that several components be present together at the cellular site of assembly; at a minimum the AAV rep and capsid proteins and the genome to be packaged. It has been proposed that trafficking networks in the producer cell direct the proteins and the genome of interest produced from the delivered plasmids back to the cellular location of the genetic element coding their production. These locations are likely not the same for each needed element. Disrupting cellular trafficking pathways and allowing random migration can be hypothesized to give more rAAV production than directed migration of each component to different locations.

In the embodiment described in this application, the disruption of intracellular trafficking is accomplished by disrupting the microtubule network.

In the embodiment described in this application microtubule disruption is accomplished by addition of vinblastine or paclitaxel to the producer cell culture.

Disrupting intra cellular communication and trafficking networks results in the production of vector that gives greater transduction in an immediate quantitative assay. The enhanced transduction must be due to some combination of more vector made per producer cell or vector particles that are on average more efficient at transduction.

More vector is made per producer cell. That, however, does not mean that the vector so made is not also a more efficient transducer on a particle-by-particle basis

Vectors made by this method transduces infected cells more gradually. This may be useful in reducing the severity of the cytokine storm that sometimes accompanies the infusion of large amount of vector into a patient.

Since the assays to quantitate the relative amounts of vector were done with cells kept in culture only briefly after infection, this slow transduction means that a larger amount of vector was being produced in the modified production than can be revealed by an immediate quantitative assays.

The vector produced by the modification transduces a percentage of the cells at levels of expression of the transgene that indicates more than one transducing vector in the cell. At the low levels of positive cells used in the assays shown herein vectors produced by the standard method rarely show multiple vectors in one cell. This means that estimating the amount of rAAV vector produced by counting the transformed cells again gives an underestimate of the amount of vector produced.

This higher than random transduction of cells by multiple vectors permits recombination between vectors of different sequences, necessary in therapies with larger genes because of the limited packaging capacity of rAAV vectors

A greater proportion of vector synthesized by the method of this application is exported from the cell making vector purification easier.

Vector made by this method has an enhanced capacity to integrate site specifically into the genome of the target cell in a Rep-protein supported manner. This phenomenon may in the future become a method for doing rAAV gene therapy. It also can serve as a marker for a better vector generally.

The enhancement to vector production found in the assays of this application was larger than one would expect, based on the work of van Lieshout et. al. and Yang et. al. . . . It is likely that the addition of disruptors of microtubule functioning contributed to increased vector yield through mechanisms in addition to increasing the interaction of the factors needed for vector assembly.

### BRIEF DESCRIPTION OF FIGURES AND TABLES

FIG. 1. A schematic of the AAV genome, which is encapsidated as a single strand. The top diagram is wt AAV showing the locations of the Inverted Terminal Repeats (ITRs), at each end of the genome, and the Rep and Cap genes, the predominant genes of AAV, between the ITRs. The bottom diagram shows a rAAV. All the sequences of the wt AAV genome have been removed except for the ITRs. The Rep and Cap genes have been replaced by the transgene and regulatory sequences for the expression of said gene, (the dashed line) also referred to as the Gene of Interest (GOI).

FIG. 2A diagram of the principle of vector production by triple transfection. Three plasmids, one containing the Rep and Cap genes, one containing the GOI inserted between two AAV ITRs, and one containing Adenovirus helper genes are simultaneously transfected into a plate of less than confluent HEK293 cells.

FIG. 3 An illustration of the putative effects of vinblastine addition on vector production. Shown as circles are the nuclei of the HEK293 producer cells. The heavy bars represent the transfected genetic elements. The dashed lines represent RNA transport out of the nucleus and protein back into the nucleus. Without vinblastine their appears to be a transport network in the cell which shuttles the proteins coded by each gene back to the region containing that gene. With the addition of vinblastine that directed transport is at least partially deconstructed. Shown here is an alteration of migration in the nucleus only, but it is likely that vinblastine affects migration everywhere in the cell.

Table 1. A comparison of the percentage of Hep G2 cells rendered positive for GFP expression upon infection with AAV-TRUF11 vector that was made either without or with vinblastine added to the producer cells. Shown are the results from using vector extracted from the cell pellet of the producer cells or exported out of the producer cells into the surrounding media. The percentages were determined by flow cytometry. The assay was performed three times on vector from the cell pellet but only once on vector from the media.

FIG. 4. A flow cytometry dot plot showing the dot plots of passage 1, from which the data of table 1, 3^{rd} set were derived. Each dot represents one cell. The diagonal line demarcates cells expressing The GFP gene from those that are not expressing GFP or expressing it at very low levels. Without the GFP gene in the cells there are not or only one or two cells to the right of the diagonal line. Also shown is a graph of the dot plot of the cells that were positive for GFP expression, i.e. cells that had been to the right of the diagonal line.

Table 2. A comparison of the percentage of HEK 293 cells, as opposed to the previous Hep G2 cells of the previous assays, rendered positive for GFP expression upon infection with AAV-TRUF11 vector made or and without vinblastine added to the producer cells. The percentages were determined by flow cytometry. #7. Designates a vector prep made without vinblastine. #12 indicates a vector prep made with vinblastine.

FIG. 5. An agarose gel comparing the amount of DNA extracted from genome containing particles produced in a vector production. Lane 7 is vector production without vinblastine. Lane 12 is vector production with vinblastine. In both cases this is material extracted from the cell pellet. SM denotes single-stranded DNA size markers, the length of which is given in kilobases. The arrow indicates a single-strand DNA genome of the length expected for the AAV-TRUF11 genome.

FIG. 6. Schematics of the number of cells on a plate during the course of infection, cell growth, and passaging. On the y axis is the amount of cells; 1X at plating, 15X at passaging. On the x axis is time,. “I” indicates point of infection. “F” indicates point of Passaging and Measurement of GFP expressing cells by flow-cytometry. The top schematic indicates infection (I) soon after passaging and replating of cells. The bottom schematic indicate an infection of confluent cells and the fate of two different plates. One plate is passaged and measured for the first time at F1, the other is not passaged then but is passaged for the first time only at F2. Not shown are plates with even later time points for the first passage. After the first passage plates are passaged and measured every 7 days (not shown).

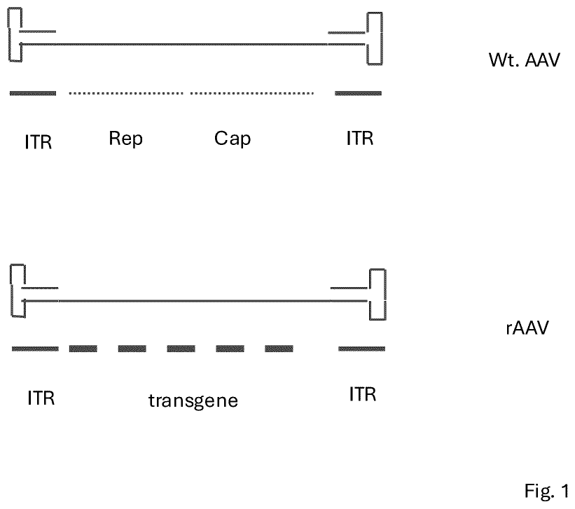

Table 3. This table shows the percentage of HepG2 cells expressing GFP on plates passaged at various times. All plates were infected at time 0. Plates were confluent at time of infection. i.e. time 0. The top row is times of passage measured as days post infection 5 days, 9 days, etc. For example: Plate V78 was passaged for the first time on day 5, for the second time on day 11, etc. Plate V79 was passaged for the first time on day 9, for the second time on day 16, etc. Plates V78 to V81 were infected with vector made without vinblastine. Plates V82 to V85 were infected with vector made with vinblastine. The lower set of values is normalization of the measurements of V78-V85.Plates V78-V81 were normalized to V78 harvested at day 5. Plates V82-V85 were normalized to V82 harvested at day 5.

FIG. 7 shows a dot plot of the flow cytometry of V78 and V82 at day 5, i.e. the first harvest and day 11, i.e. the second harvest of each of these plates. Plate 82, the plate infected with vinblastine made virus has more GFP expressing cells than does Plate 78, the plate infected with non-vinblastine made virus.

FIG. 8 shows a comparison of six plates from the day 23 harvest. V79 and V83 are from their 3^{rd} harvest. V80 and V84 are from their second harvest. V81 and V85 are from their first harvest. In all cases the plates infected with vinblastine made virus, V83, V84 and V85, have more GFP expressing cells than do the matching plates infected with the non-vinblastine made virus, V79, V80, and V81.

Table 4. A table showing the fold reduction in the percent of GFP positive cells between the first passage and the second passage for plates V78 to V85. Table was derived from the data of Table 3.

Table 5. An assay as of the same sort as Table 3. In this case initial percentage of GFP expressing cells between infections made with or without vinblastine were more similar. Plates V86 and V87 were infected with vector made without vinblastine addition. V88 and V89 were infected with vector made with vinblastine addition. The percentages were determined by flow cytometry.

Table 6. An assay as of the same sort as Table 3 except that now the cells have been plated on Matrigel to reduce the ability of HepG2 cells to continue dividing after reaching confluence. Lane 7 is infection of Hep G2 cells with vector made without vinblastine addition. Lane 12 is infection with vector made with vinblastine. The percentages were determined by flow cytometry.

The bottom rows shows plates that were harvested sooner after transfection.

Table 7. A demonstration that Hep G2 cells infected with vinblastine made vector have a greater percentage of cells expressing higher levels of GFP. V1, V2, V5 are infections of vector made without vinblastine. V3, V4, V6 are infections with vector made with vinblastine. The percentages and the number of cells at each brightness level were determined by flow cytometry at the first passage, days 10 and 17 respectively. FITC Bins are cells with different levels of GFP brightness. Bin1 are the brightest cells as determined by flow cytometry, Bin 2 are the next brightest, etc. These plates were infected with vector extracted from the producer cell pellets.

Table 8. A demonstration that in plates infected with cells from the media fractions there are also more bright cells when the infection was performed with vector from the vinblastine production, i.e. #12, than when cells are infected with vector produced without vinblastine addition, i.e. #7.

Table 9. A comparison of the percentage of Hep G2 cells rendered positive for GFP expression upon infection with AAV-TRUF11 vector made with and without paclitaxel added to the producer cells. The percentages were determined by flow cytometry.

FIG. 9 Shows a dot plot from a flow cytometry of the first and second passages of the two infections of the data of Table 9.

Table 10. A comparison of the percentage of Hep G2 cells rendered positive for GFP expression upon infection with AAV-TRUF11 vector made without vinblastine, made with vinblastine at two different levels, or made with paclitaxel. The percentages were determined by flow cytometry.

Table 11. A comparison of the percentage of Hep G2 cells rendered positive for GFP expression upon infection with AAV-TRUF11 vector made with paclitaxel alone or with both paclitaxel and vinblastine together. This demonstrates a lack of synergy within the producer cells with reference to vector production.

FIG. 10. This figure shows flow cytometry dot plots from two parallel infections of Hep G2 cells several passages after infection. One infection was with vector made with vinblastine and the other infection was with vector made with paclitaxel. Also shown is a dot blot of an infection performed with vector that did not have either vinblastine or paclitaxel at infection, F147, which gave an exceptionally good cluster. All three plates were coinfected with wt AAV.

FIG. 11 shows an assay done with one plate receiving vector made without vinblastine; there is little sign of site-specific integration on this plate. The cells on the other plate were infected with a vector made with vinblastine; on this plate there is a large population of cells whose higher expression level is consistent with site-specific integration or multiple vectors. Both plates were coinfected with wt AAV. The flow cytometry on this assay was performed at their first passage.

FIG. 12 shows graphs from flow cytometry of cells infected vector that are positive for GFP expression with the percent of positive cells for each plate also shown. From left to right: first infection with a non-vinblastine made vector and coinfection with a wt virus, second an infection with a vinblastine made vector but without a coinfection with wt virus, and third infection with a vinblastine made vector and coinfection with wt virus. The peak of expression for cells that have infection at the AAVS1 site is approximately 10⁴.

Table 12 shows a comparison of the percentage of Hep G2 cells rendered positive for GFP expression upon infection with AAV-TRUF11 vector made with and without vinblastine, These two vectors were made in HEK293 cells with transfection by PEI rather than the normal CaCl₂. Vinblastine was added 2 hours post transfection, when the media was changed. The percentages were determined by flow cytometry.

FIG. 13 shows a comparison of the percentage of Hep G2 cells rendered positive for GFP expression upon infection with AAV-TRUF11 vector made without vinblastine and with vinblastine added at two different times as specified in the table. These two vectors were made in HEK293 cells with transfection by lipofectamine rather than the normal CaCl₂. Also shown is a graph of this data. “M” designates cells infected with vector from the media of the producer plates; Data not designated with an “M” is infection with vector from the cell pellet. The percentages were determined by flow cytometry.

### DETAILED DESCRIPTION OF THE INVENTION

In the use of rAAV as a vector for gene therapy, a limiting factor is the difficulty of producing high quality vector in large quantities. Herein is descried a modification to present rAAV production methods to improve rAAV vector production.

### Glossary

“Vector” refers in this application to a virus that is being used to transport genetic elements, i.e. DNA or RNA, into cells. In the data shown in this application the word “vector” refers to recombinant adeno associated virus (rAAV). However, vectors are produced from other viruses, both closely related and unrelated, and the methods described in this application are likely to benefit in the production of non rAAV vectors also. In the rAAV vector almost all the AAV genome has been removed and replaced with exogenous sequences.

“Transfection” versus “infection”. Transfection is the process of driving plasmids into cells, in this application into HEK293T cells, the producer cells for vector production. Infection is performed only with wt AAV or viral vectors, in this application the vector made in the producer cells. Since the vector has a viral coat it is able to enter a cell as a virus does, i.e. without manipulation of the cell envelope.

“Uncoating” is being used in this application as a stand in for the expression of the transgene in the rAAV infected cell. It is the protein's function that is being measured. As such uncoating involves numerous steps beginning at infection and up to production of the protein coded by the genome contained within the delivered vector, in this case the GFP protein. When a vector is described as uncoating more slowly, it is any one of these steps that could be occurring at a reduced rate, not necessarily the specific step of release of the genome from the vector particle.

The term “Transduction” herein is used to describe the introduction of exogenous genetic material into the cell by a viral vector and its subsequent expression and resultant protein production. What is being measured are the percent of cells in the targeted population that express this protein and in some cases the amount of protein.

The term “transduction capacity” refers to the number of vector particles produced and the percentage of those particles that can transduce a target cell. Determining this is in general made difficult by a vector infecting different cell types with more or less effectiveness and also by different capsids infecting different cell types with more or less efficiency. In this application only one capsid is being used and with one exception only one cell type is being targeted making assays of comparative effectiveness more doable. It would be simple to compare the improvement in vector production by merely counting the number of cells that express the GFP protein after infection. However, that gives a substantial undercount to the number of transducing-able particles this method gives. As illustrated below, there are two reasons for this. First the vector produced by this method transduces cells more gradually than the vector produced by the standard method. Since these were brief assays, this leads to an undercount. Second the vector produced by this method seems often to transduce a target cell with more than one vector as opposed to the vector produced by the standard method, which has no such tendency. This multiple infection is a very useful property but again counting GFP expressing cells leads to an undercount of particles produced. The question of how much of an increase in transduction capacity is due to an increase in particle number and how much is due to better made particles is unresolved.

“Trafficking and communication” is a phrase used herein to describe movement of molecules within the cell. Trafficking and communication cannot be thought of as separate phenomena, since communication within cells is most often accomplished by trafficking of specific molecules.

“wt AAV” refers to wild-type AAV. This is the natural form of the AAV virus, in this case AAV2, i.e. without any modifications to its genome. Coinfection with wt AAV was performed with infection by the AAV-TRUF11 vector in many of the assays.

It should be understood that by the designation “AAV” and “rAAV” we are referring to the entire catalogue of existing adeno-associated virus and the recombinant adeno-associated virus that can be derived from them as well as those that will be developed in the future. These include the known AAV serotypes: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), as well as modified AAVs and non human AAVs; AAVbb2, AAVcy5, AAVrh10, AAVrh20, AAVrh39, AAVrh43, AAVrh64R1, AAVhu37 avian AAV, bovine AAV, canine AAV, equine AAV, primate AAV, non-primate AAV, ovine AAV. It also includes AAV comprising a capsid protein of one AAV subtype and genomic material of another subtype, and AAVs with mutant or chemically modified capsid protein, or AAVs in which the capsid is chimeric i.e. an AAV capsid with regions derived from more than one AAV serotype or other parvoviruses. It is also meant to include the closely related, but non-AAV parvoviruses or sequences from these parvoviruses, e.g. bocaviruses, goose parvovirus, etc.

### Standard Method of Vector Production

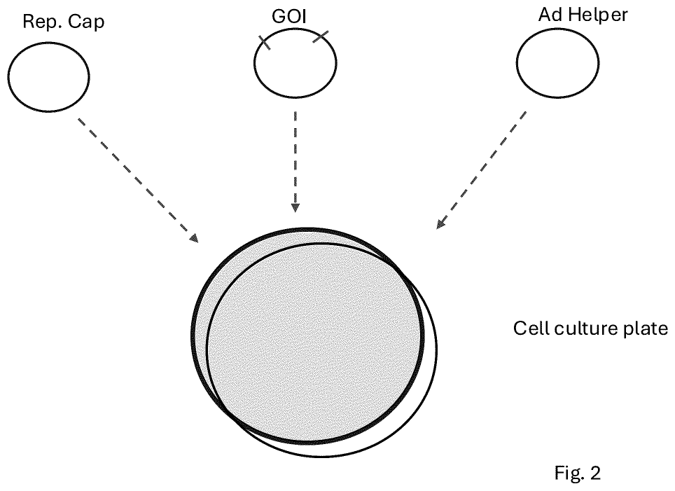

The standard method of rAAV production is referred to as the triple transfection method (FIG. 2). In this method the most common producer cells, are from a human embryonic kidney cell line referred to as HEK293 (or HEK293T if they contain the simian virus 40 T-antigen gene. In this embodiment it is HEK293T that is used.). There are modifications to HEK293 cells being tested to increase their ability to produce rAAV vector. Other cell types are also being investigated for rAAV vector production such as Chinese hamster ovary cells and yeast cells. It should be understood that the use of HEK293T cells is just one embodiment of this invention and the invention can be used with other cell types.

The producer cells are simultaneously transfected with three plasmids containing the genes needed for the HEK293T cells to produce the viral vector. One plasmid contains the gene of interest (GOI) and regulatory sequences for this gene. The GOI and regulatory sequences are inserted between two AAV inverted terminal repeats (ITRs). This structure ITR-gene of interest-ITR will be separated from its plasmid backbone in the producer cell, a function referred to as “rescue” and will become packaged in the viral capsid as the genome of the produced vector. A second plasmid contains the Rep and Cap genes of AAV: the Rep gene playing a role in all viral functions such as DNA replication and encapsidation of the genome into the completed particle. The Cap gene encodes the AAV capsid protein. The third plasmid contains 6 genes from adenovirus needed to render the HEK293T cells permissive for rAAV vector production. (As mentioned above the AAV viruses are not autonomous, i.e. they are not able to replicate on their own after infecting a cell. They require that the cell they infect be coinfected with another virus, such as adenovirus, hence the need for supplying adenovirus genes for rAAV production.) Some newer methods of vector production have been developed such as the use of baculovirus and SF9 cells. (U.S. Pat. No. 7,271,002). These methods may also benefit by the methods of this application.

Evidence has been reported showing that one reason for poor yields of recombinant adeno-associated virus vectors is that apparently the proteins necessary for vector production, coded by the Rep and Cap containing helper plasmids, were, after synthesis, directed back to the site of synthesis of the mRNA coding for these proteins. While this is efficient for production of wt AAV, it is counterproductive for synthesis of complete rAAV particles which are likely not being produced in the cellular location that the mRNA for the Rep and Cap proteins is produced.

Several more recent reports have made observations consistent with this possibility.

Nonnenmacher and Weber in explaining why in “directed evolution” methods of generating new capsids, they did not find that capsids were packaging heterologous genomes present in the same producer cell. They found instead a preferential incorporation into capsids of the genome that encoded that capsid.

Van Lieshout et. al have found, in making vector with the same multiple transfection approach, that when the Gene of interest construct and the Rep Cap genes were combined on one plasmid, thereby making vector production a double transfection, the yields of vector were greater than when doing the standard triple transfection. (It had been feared that such a large plasmid that also contained AAV ITRs would be hard to manufacture in high quantities and poorly transfectable.) This plasmid combination was superior to the other possible dual plasmid transfections in which two gene sets were combined into one plasmid. They also found that this gave vector with a lower percentage of partially packaged genomes and of empty particles. A vector so made containing a phenylalanine hydroxylase gene, when injected into mice, gave phenylalanine levels equivalent to those obtained with vector produced by standard triple transfection.

Both these results were consistent with the model of Ward et al which proposed that in rAAV viral production the AAV proteins that are required for vector production were being directed back to the site of replication and transcription of the coding genome.

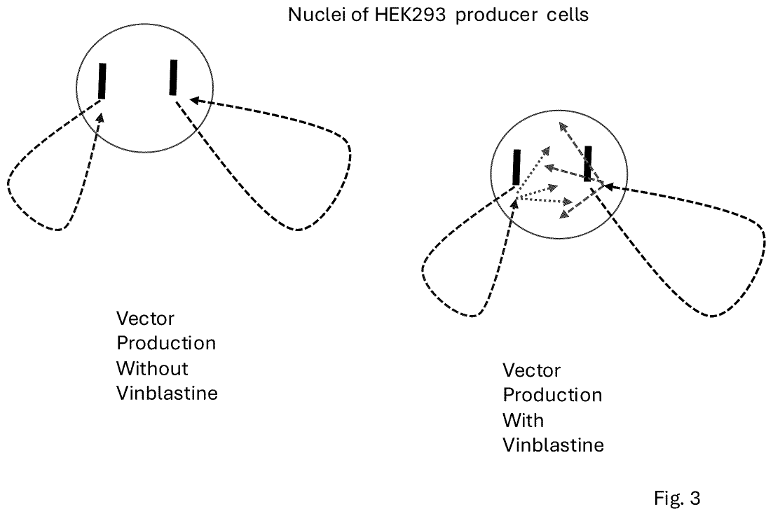

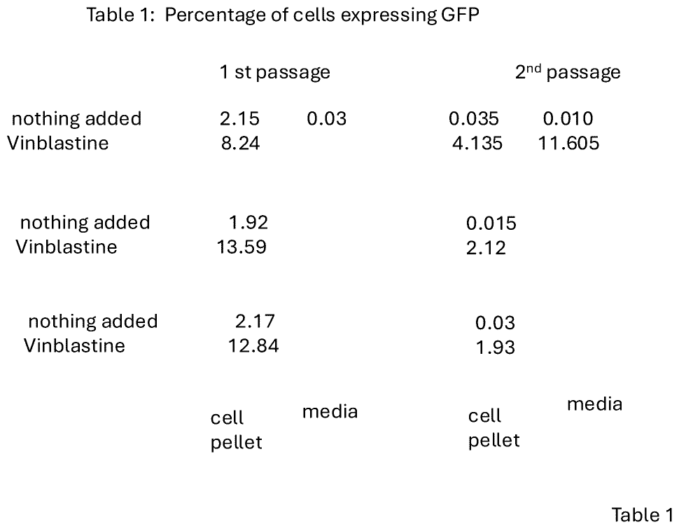

This application describes a method to disrupt this transport, in which the Rep and Cap proteins are likely randomly distributed in the cell rather than specifically directed to an inopportune location, FIG. 3. The method of this application consists of disrupting intracellular transport and communication by addition of inhibitors of microtubule polymerization and depolymerization during viral production. Disruption of normal cellular transport mechanisms would seem counterproductive, but while it must hinder most cellular processes, it proves useful in the production of rAAV.

### Method of Viral Production. (Protocol)

### Triple Transfection Method of Viral Production.

Producer cells, i.e. a human embryonic kidney cell line designated HEK293T, (derived from HEK293, ATCC No. CRL1573, with the addition of the gene for SV40 T Antigen), are simultaneously transfected with three plasmids, one containing the needed AAV genes, i.e. the Rep and Cap genes, one containing the necessary adenovirus helper genes, for example pXX6 and one containing the desired transgene flanked by AAV viral ITRs, in this case pAAV-TRUF11. (Some groups use a plasmid that combines the Rep, Cap and adenovirus genes on the same plasmid, a plasmid named pDG.) In this application the AAV-TRUF11 genome is the gene of interest and is being used to produce an rAAV. (the AAV-TRUF11 construct, probably the most widely used genome in AAV gene therapy studies, contains an enhanced green fluorescent protein, GFP, driven by a chicken-beta-actin cytomegalovirus promoter between two AAV ITRs. It also contains a Neomycin resistance gene, however, at no time during these assays were cells treated with G418.) The recombinant virus, AAV-TRUF11, was produced by co-transfection of equimolar amounts of pAAV-TRUF11,, pXX6, and an AAV2 Rep-Cap construct into non-confluent HEK 293T cells. Transfection was mediated by the standard CaCl₂ protocol, as described below. Cells were harvested at 48-72 hrs. post transfection by squirting the plates with the media on the plates. The mix of cells and media was centrifuged and the media preserved for measuring the vector it contained which was not cell associated. The cell pellet was resuspended in TE, frozen and thawed 3X, then digested with benzonase nuclease. The cell pellet contains the vector remaining in the cell at harvest.

Wild-type AAV2 was produced by infection of HEK 293T cells with virus produced from pAV2 (or by transfection of the plasmid pAV2.) and transfection of pXX6. The viruses were stored at −80 degrees.

It should be noted that academic labs and commercial entities that produce rAAV use a great variety of plasmids for these three functions. The differences between the plasmids used in triple transfection are, except for the Gene of interest, minor; all the plasmids are thought to function in the same manner.

### Detailed Protocol.

HEK 293T cells were plated in DMEM with 10% Fetal Bovine Serum (FBS) and allowed to grow until the plate was from 40-60% confluent, generally overnight. Media was replaced with low glucose DMEM with 2% FBS.

Plates were incubated for 2 hours in this media, then the transfection mix was added. The three plasmids (pAAV-TRUF11, pXX680, and p6-7) were combined in one tube at amounts that are approximately equimolar. The plasmids are approximately the same size, therefore equal amount by mass can be used; e.g. approximately 0.8 ug per well of each plasmid in one well of a 6-well plate. One well of a 6-well plate holds typically 2.0 mls of media. (All transfection protocols described herein are using 6-well plates.) More commercial applications will necessarily scale up this protocol.

Add water to plasmid mixture to bring volume to 15 ul

Mix

10.0 ul CaCl₂ (2.5M)

Mix

75 ul sdH2O. i.e. bring up to 100 ul

100 ul HeBS. Add slowly drop by drop with continuous swirling

HeBS composition:

50 mM Hepes

280 mM Na Cl

1.5 mM Na₂HPO₄

(pH adjusted to 7.1 with NaOh)

Bubble air through mix at least 20×.

Let mixture sit before adding to cells. 15′ (this is slightly longer than is usually suggested)

Mix mixture before putting on cells

Add to cells slowly drop by drop with gentle swirling of the plate

In the Only Deviation from the Above Standard Method:

Vinblastine (an antitumor alkaloid; International Chemical identifier C46H58N409) was added to the plate of HEK293T cells 90 minutes prior to transfection. Vinblastine was added to a concentration of 100 nM. The timing of vinblastine addition was not limited to 90′ prior to transfection as vinblastine could instead be added after transfection with similar results.

(The cells after this treatment are more distorted than after a normal transfection i.e one without added vinblastine. As a general rule higher amounts of vinblastine result in greater amounts of virus but adding vinblastine to a higher concentration than 100 nM is counterproductive in causing too much cellular disruption. The optimal concentration of vinblastine will most likely need to be determined uniquely by each production facility due to variance between HEK293 strains and variations on the transfection method and cell culture volumes used.)

Harvest at 48-72 hrs. post transfection.

Dislodge cells by squirting with media

Spin 4 min at moderate speed e.g. 7500 RPM

Remove supernatant. Store at −80 degrees. Later will measure “media” virus directly from this tube.

Resuspend pellet in 150 ul TE

Freeze/thaw 3×

Pipet a few times after each thaw to partially disaggregate clumps.

After 3^{rd} thaw, added:

6.0 ul 3.0 M Nacl

4.0 ul 100 mM MgCl

0.8 ul 1.0 M tris pH. 7.8

2.0 ul benzonase at 1 unit/ul. No DNAse

37 degrees for 10 min

Remove and use pipettman to completely disperse lumps

37 degrees for 4 hours

Store at −80 degrees.

Infection protocol for comparing rAAV vector made with vinblastine with rAAV vector made without vinblastine.

Hep G2 cells (Hep G2 is a standard cell line isolated from a human hepatocellular carcinoma) were grown in DMEM in 24-well plates supplemented with 10% fetal bovine serum. Media was changed immediately prior to infection. (This media change is not required for this protocol.) Upon infection cells were maintained in DMEM and 10% FBS with Penicillin (50 units/ml) and Streptomycin (50 ug/ml). Media was changed after 5 days. Plates were first passaged 7 days post infection except where otherwise noted. Subsequently cells were passaged every 7 days, except when otherwise noted, with their media replaced on the 5^{th} day after each passaging. Cells were split at 15:1 at passaging. Therefore, when desired, as many as 14 duplicates plates can be made at passaging. With such a high passage ratio, by the second passage post infection, non-integrated GFP expressing genomes have been diluted by cell division or lost by nuclear membrane dissolution and reformation. We chose to use a level of AAV-TRUF11 virus that resulted in a low percentage of the population expressing GFP, as determined by flow cytometry.

Cells were processed for flow cytometry on a Becton Dickinson FACS Canto flow cytometer using FACS Diva software. Cells were determined to be positive for GFP expression by FITC/PE ratio as described previously. When plotting FITC vs. PE in flow cytometry, cells whose greenness is derived from endogenous sources have a defined PE/FITC ratio when graphed. This ratio is different for the GFP protein. In other projects dozens of diverse chemicals, including vinblastine, have been added to Hep G2 cell culture. A chemical addition that gave a PE/FITC ratio that produced Hep G2 cells that strayed into the GFP expressing region of the graph, when no GFP gene was present, was never observed. On graphs in which the cytometer is plotting counts vs FITC, the Y-axis (i.e. the counts axis) was showing the numbers of cells at vatious levels of FITC expression.

### Effects of Vinblastine Addition.

Plates of growing Hep G2 cells were infected with the produced rAAV vector, AAV-TRUF11. The percentage of the total volume of the vector preparation from the producer cell pellet that was used for infection was one third the percentage of the total volume of the vector preparation from the media. This 3-fold difference was based on anticipated results. In a normal vector preparation of AAV2 based rAAV, little vector is exported to the media. This turned out not to be the case with vector made with vinblastine. In most later infections equivalent percentages of the pellet and the media preparations were used. For example, if the total volume of material from the cell pellet was 150 ul and the total volume from the media was 450 ul, then if a plate was infected with 2.0 ul from the cell pellet, the parallel plate from the media fraction was infected with 6.0 ul of material. In this way one can determine approximately the relative amounts of vector that had remained in the cell compared with vector that had been exported to the media. The plates in this assay were also simultaneously coinfected with wild type AAV2. Wild type AAV2 coinfection along with a rAAV infection results in some small fraction of the cells that take up rAAV integrating the rAAV genome into the host cells at a region of chromosome 19 known as AAVS1. No other difference between cells with or without a simultaneous infection with wt AAV was observed in these assays.

Table 1 illustrates that the addition of vinblastine to the production of the AAV-TRUF11 vector, as described above, results in vector that gives a higher percentage of transduced cells than does infection with vector produced in parallel but without the addition of vinblastine. Compare 2.15% and 8.24%. Shown are the percentage of transduced cells from both the first passage of the Hep G2 cells and from the 2^{nd} passage, ie 7 days later after a 15:1 split and regrowth of the cells. Compare 0.35% to 4.135%. Transduction requires that vector particles can infect the target cell, have their genome transported to the nucleus with subsequent transcription into RNA. This RNA must then be exported to the cytoplasm and translated into protein. If any of these steps cannot be accomplished, transduction will not be measured. The conclusion is that the addition of vinblastine to an rAAV production results in some combination of either a greater number of complete vector particles or vector particles which are more effective at transduction. The second and third sets of assays were done with the same virus preps. The first two sets of assays were performed with wt AAV added to the infection of the Hep G2 cells. The third assay was done without wt AAV addition.

Shown, in the first set, are the transducing percentages from both the material derived from the cell pellet and from the media. In the latter two sets, the assay on virus from the media was not repeated. As seen in table 1, most of the transduction capacity using the vector made without vinblastine remained in the cell pellet. In contrast much. of the transduction capacity from the infection done with vector made with vinblastine is found in the media. (As mentioned above in this assay there is 3-fold more material from the media fraction applied to the cells as from the cell pellet fraction.) There is no measurement shown for the first passage of cells from the media fraction from the vinblastine plate to avoid the possibility that the presence of some vinblastine might affect expression. A conclusion is that a greater percentage of the virus produced in the vinblastine treated cells is exported out of the producer cells.

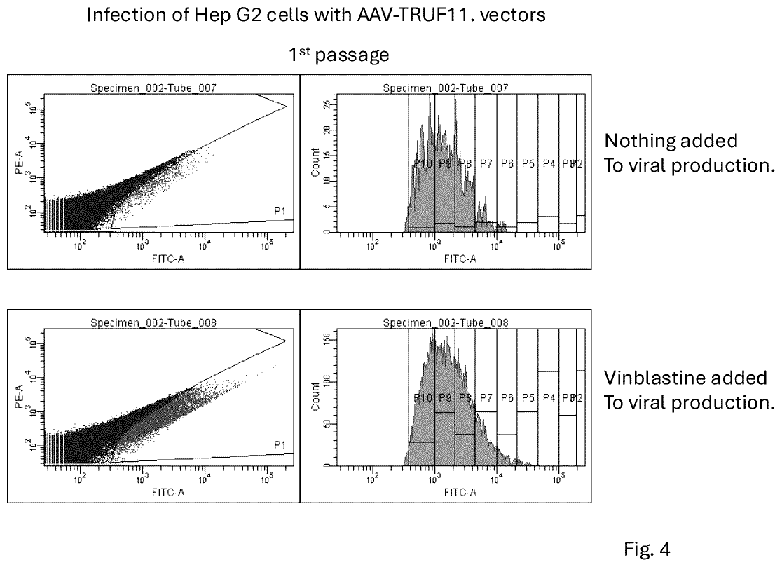

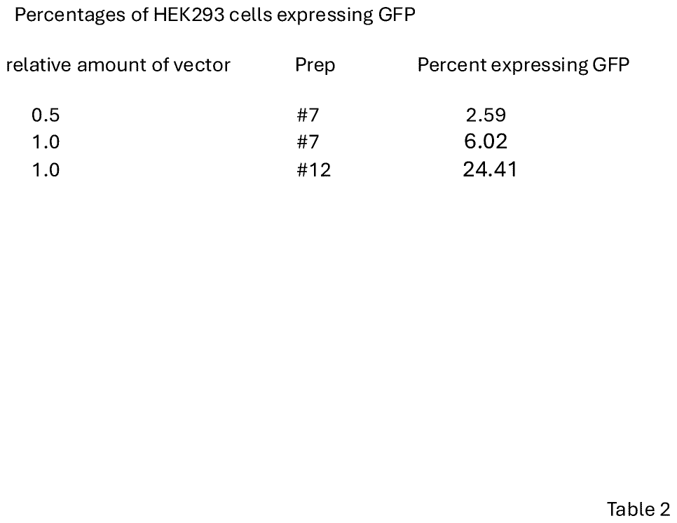

FIG. 4 shows the flow cytometry from which the numbers in the third experiment of Table 1, (the pellet fraction at the first passage) were derived. In each assay FITC expression from 100,000 cells was measured. Each dot on the flow diagram, the left panels, represents 1 cell. The GFP gene in the AAV-TRUF11 virus codes for a protein that fluoresces green when irradiated with UV light. The diagonal line in the diagram demarcates the cells that are expressing the GFP protein from those that are not. Cells to the right of the line are expressing the protein, to the left they are not. Without infection of the cells with the AAV-TRUF11 virus there are no or at most one or two cells to the right of the dividing line. (This was a consistent result seen in hundreds of assays on other unrelated projects.) Some cells that express low levels of the GFP protein will be to the left of the line because more of their UV fluorescence is still derived from endogenous elements of HepG2 cells than from the GFP fluorescence. The right panels are graphs of the cells that scored positive in the flow cytometry. i.e. cells that were to the right of the line on the left panels.

The cells infected by the two vector preparations differ in several further respects. As shown in table 1 there are more GFP expressing cells in the infection with vector made with vinblastine in their production but in addition the number of GFP expressing cells in the plate infected with vector made without vinblastine falls more sharply with growth and passage than the number of GFP expressing cells in the plate infected with vector made with vinblastine. Second, the percentage of cells with higher GFP expression levels (i.e. cells that are further to the right on the graphs of FIG. 4) is somewhat higher in the plate infected with vinblastine produced vector than in the other plate. These additional differences are dealt with below.

Table 2 shows higher transduction from the vinblastine made vector when the infections are of an alternate cell line, in this case HEK293T cells, i.e. the same cell line used for vector production. HEK293T cells were infected with material from the producer cell pellets then harvested one day later to minimize the loss of episomal units from cell division. The vinblastine-made vector resulted in 4× as many cells demonstrating GFP expression as did cells infected with vector produced without vinblastine addition during their production.

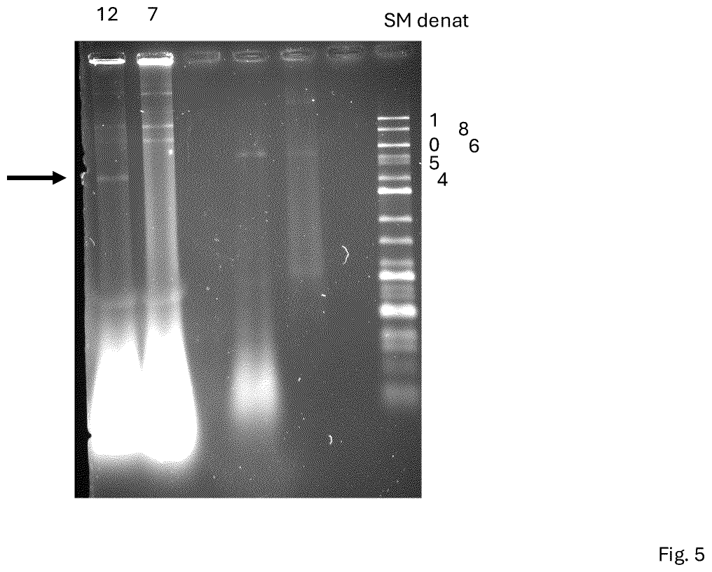

An examination of genome containing particles shows that when the vector is made with vinblastine the amount of genome containing particles is greater when compared with vector made without vinblastine. FIG. 5 shows by agarose gel electrophoresis the relative amounts of vector genomes extracted from genome containing particles in vector made without vinblastine, #7, in comparison to vector made with vinblastine, #12,. 10% of the resuspended cell pellet from a 6-well plate (a volume of 15.0 ul) was treated as follows:

15 ul of treated resuspended pellet

Added in order:

1 ul prot k

2 ul 0.25 M EDTA (ethylene diaminetetraacetic acid)

1 ul 10% sds. (sodium dodecyl sulfate)

37 degrees 4 hr.

Stored o/n at −20 degrees

Thawed

Added:

3.0 ul 1.5 N NaOh

37 degrees 30′

add glycerol and dye

For size markers in this electrophoresis, commercial double-stranded DNA size markers were used. Since the genomes released from the lysed viral particles will be single-stranded, denature size markers as follows.

Bring up to to 10.0 ul

1.5 ul 1.5 N NaOh

98 degrees for 4′

electrophorese on a 1.2% agarose gel.

Stain with SYBR Gold. (Thermo Fisher Scientific)

The proteinase K destroys the benzonase nuclease from the vector preparation so that the vector DNA will not be digested when the vector particles are lysed by NaOh. The cell pellet material includes both the “genome containing particles” and vector particles without encapsidated genomes or with only partial genomes. The NaOh will lyse all vector particles. SYBR Gold will stain any RNA or DNA present on the gel whether double-stranded or single-stranded. The complete vector genomes that had been contained in these particles can be identified and distinguished from other nucleic acids present in the cell because they electrophorese at slightly less than 5 KB, unlike anything else present in the producer cells, e.g. the transfected plasmid DNA. The bright stain at the bottom of each lane is benzonase digested cellular and plasmid DNA. Determining the relative amounts of genome containing particles, (i.e. capsids containing a complete vector genome) by this method eliminates the possible causes for incorrect values due to incompletely digested plasmid DNA that often corrupts measurements by hybridization and Polymerase Chain Reaction (PCR). Incompletely digested plasmids contain all the sequences present in the rAAV genome and so would be scored by hybridization. Measurement of the DNA in genome containing particles by PCR and hybridization will also score positive for incomplete genome fragments that are encapsidated.

As shown in FIG. 5, there is a band, designated by the arrow, of the correct size in #12, the vector made with vinblastine; a similar band in not present in #7, the vector made without vinblastine. The conclusion from this assay is that there are more genome containing vector particles produced when vinblastine is added to the producing cells.

This measurement is only of the vector found in the pellet, which forms the bulk of the transduction capacity in #7 but only about half of the transduction capacity of #12. Consequently, the difference in relative amounts of total encapsidated genomes is likely underestimated in this measurement of only the encapsidated genomes from the cell pellet. Importantly, this assay does not imply that some of the increased transduction capacity of the vinblastine made vector might not derive from that vector being a more efficient transduction agent on a particle-to-particle basis than vector made without vinblastine.

The vector made with vinblastine uncoats more slowly in the infected cell than does the vector made without vinblastine. We had observed by visual inspection of infected plates that on both dividing cell populations and on confluent cell populations (therefore dividing less robustly) GFP expressing cells appeared more rapidly on the plates infected with #7 vector than those infected with #12 vector. To investigate this the following assays were performed.

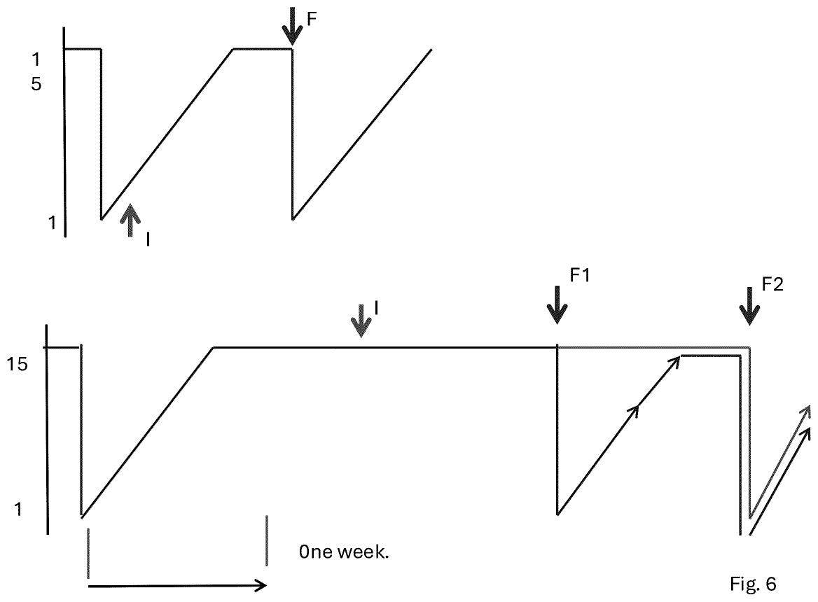

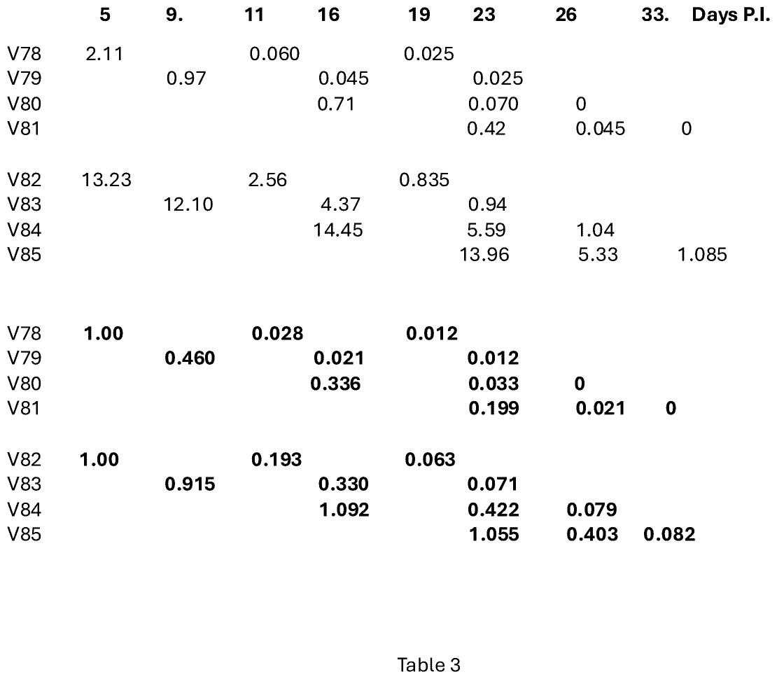

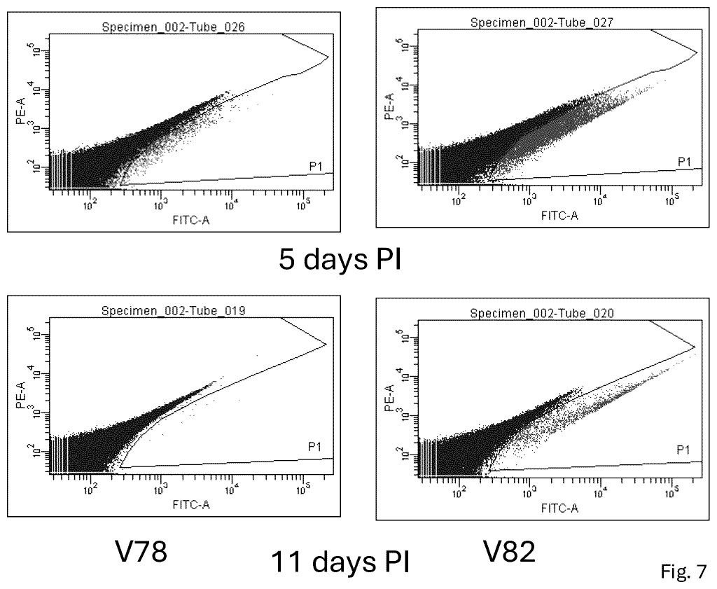

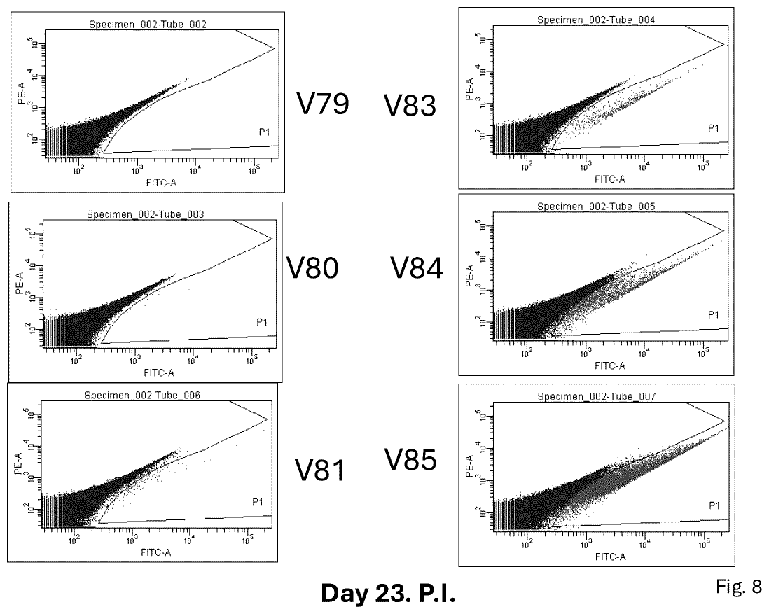

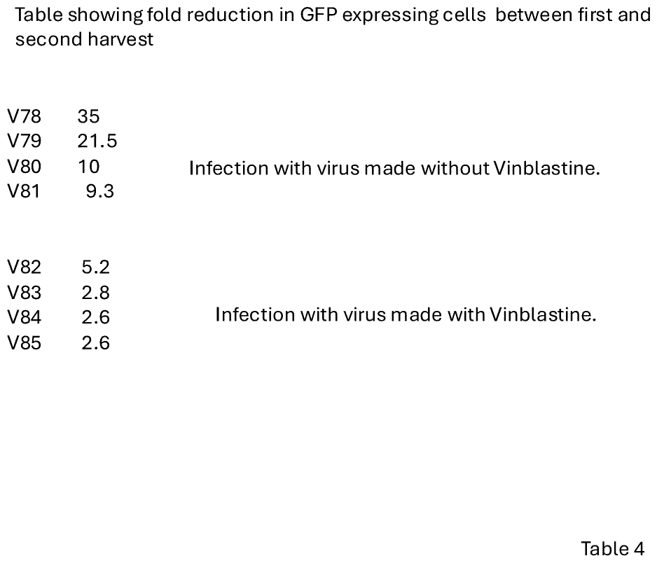

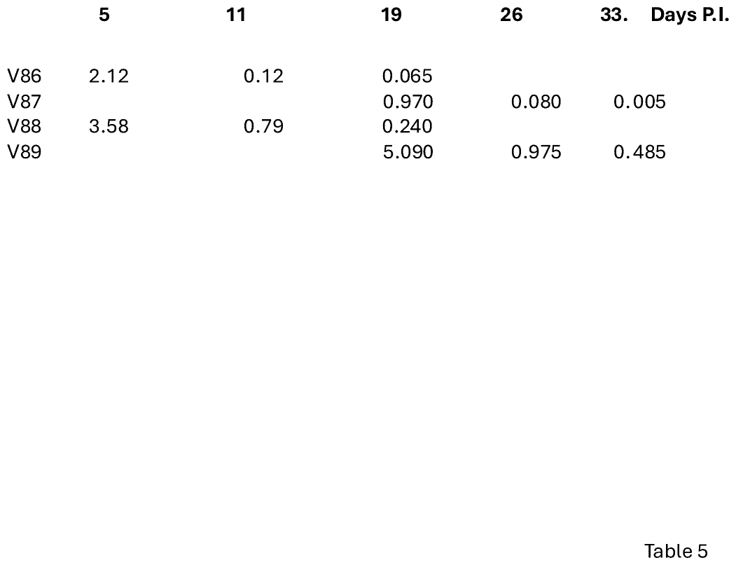

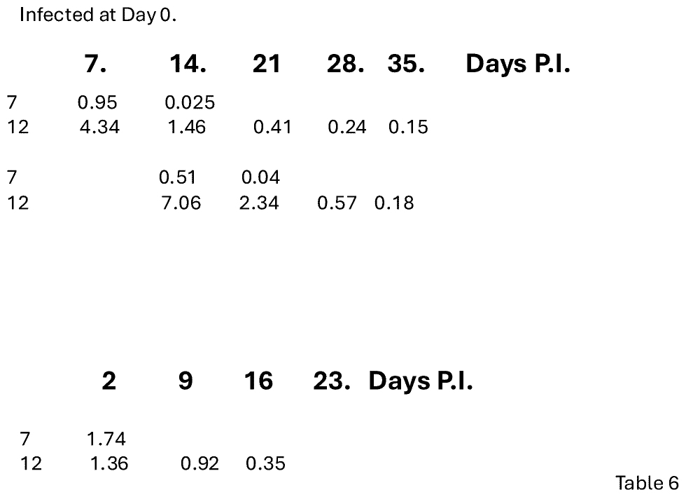

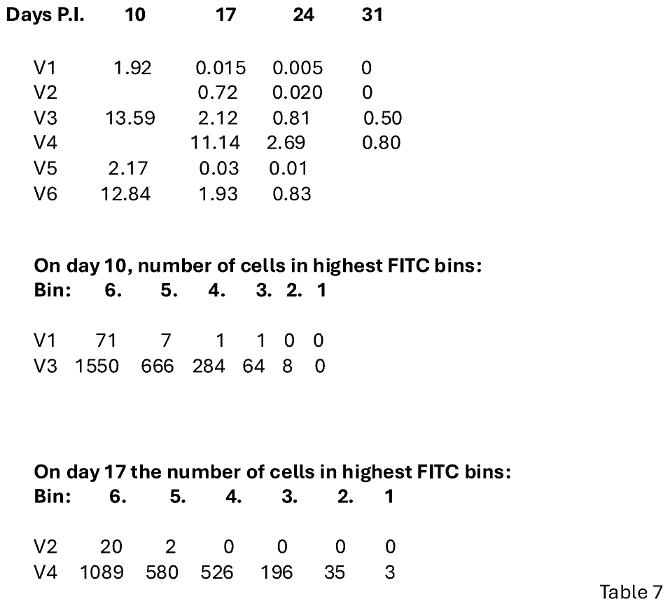

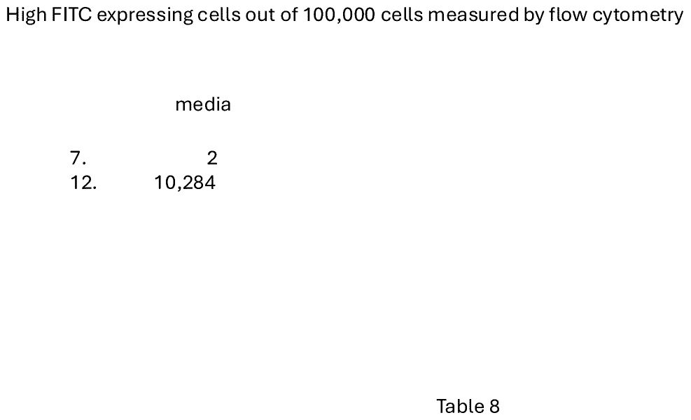

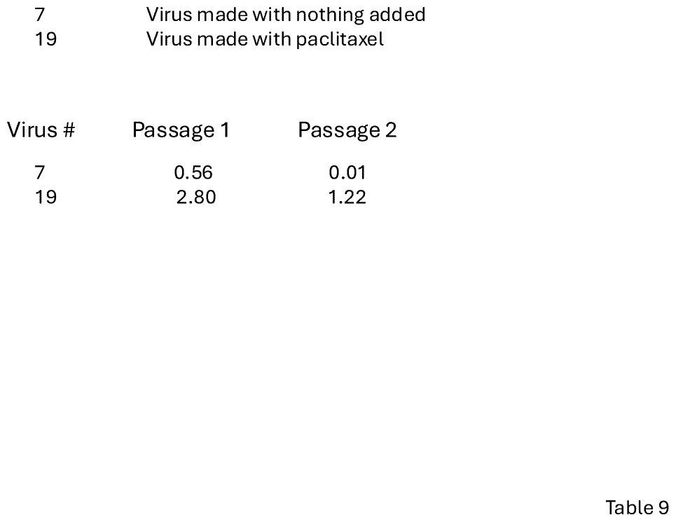

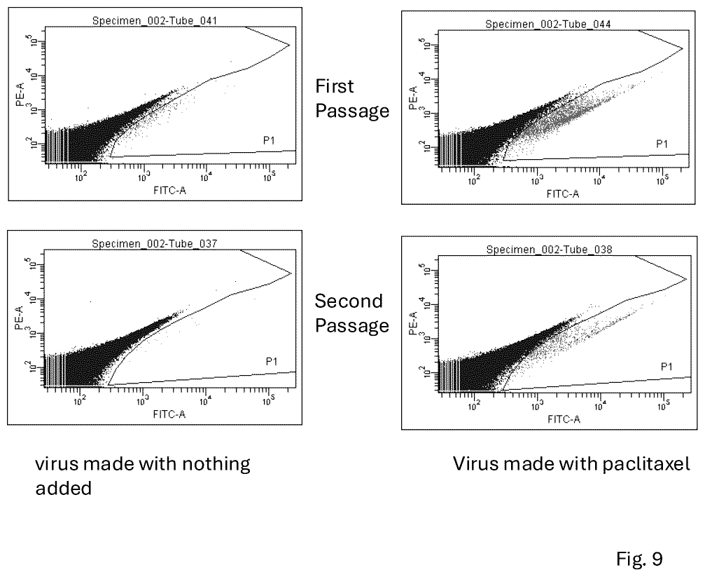

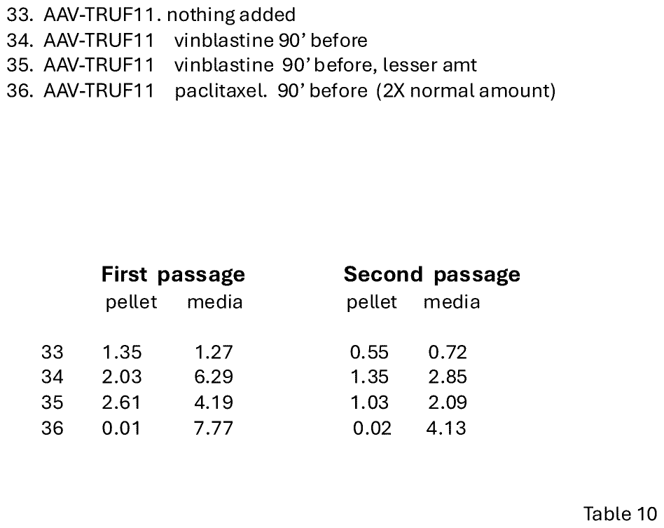

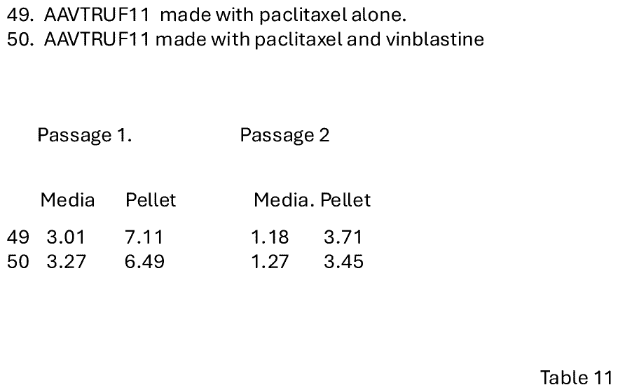

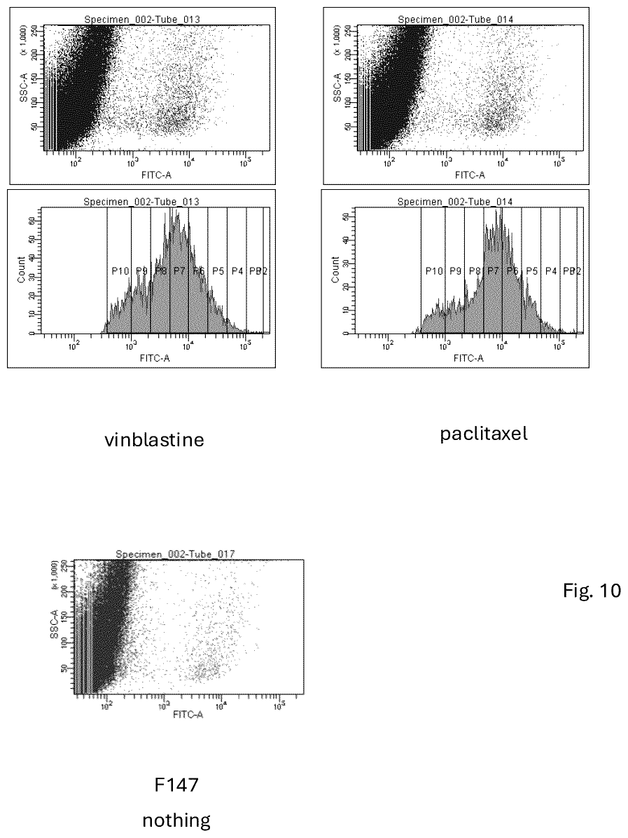

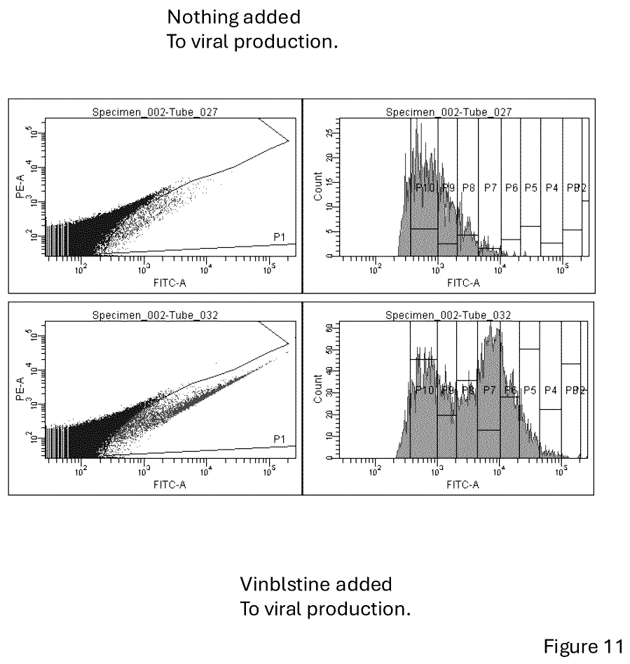

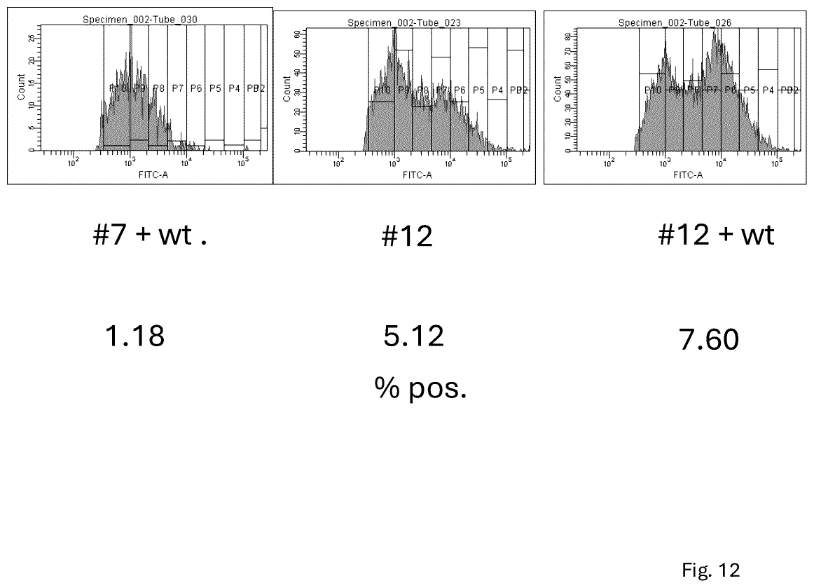

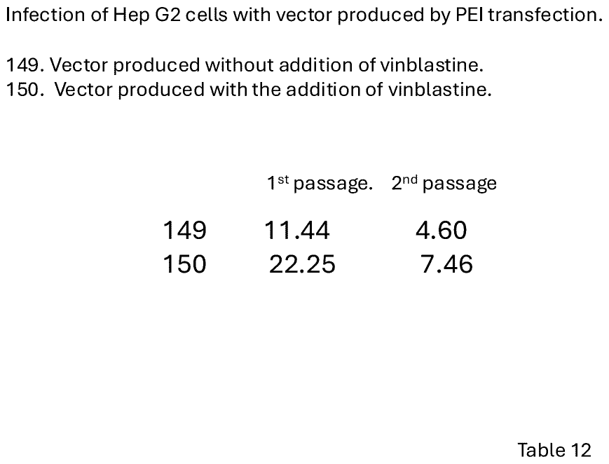

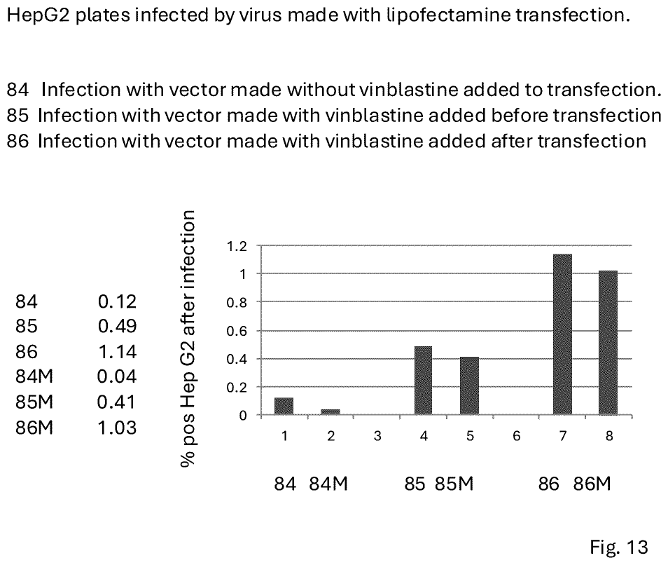

FIG. 6 is a schematic illustrating the timing of the sort of assay that follows. The y axis represents the relative number of cells on Hep G2 plates being used to measure the transduction of vectors, i.e. from 1 to 15, with 15 being confluence and 1 being the relative amount of cells after 15:1 passaging. The x axis shows time. Normally, as shown in the top schematic, the cells are split 15:1 every 7 days “I”. indicates infection, “F” indicates Passaging and measurement by Flow Cytometry. So shown in the top schematic is infection 1 day after the plate is passaged and passaging and flow cytometry a day or so after the plate reaches a relatively confluent state. The bottom schematic shows infection of parallel plates in which the cells have attained confluence before infection, infection occurring at I. One parallel plate is passaged for the first time at F1, the second plate, also infected at I, continues in culture and is passaged for the first time at F2. In the assay below there were two sets of four plates each, with each of the plates in a set having its first passage at 4 different, ever later times, i.e. as if at F1, F2,F3,F4.

Table 3 shows the results from infections and passaging of plates with a schedule as seen in the bottom schematic of FIG. 6. However, in the assay of table 3 there are not merely 2 parallel plates but 8 parallel plates; 4 plates infected with vector made without vinblastine and 4 plates of cells infected with vector made with vinblastine.

The eight wells of confluent Hep G2 cells were simultaneously infected with vector extracted from the cell pellets. (The lower amounts of vector exported to the media in the non-vinblastine producers precluded such assays with vector from the media.) Four wells were infected with vector from a vector production in which vinblastine was not added, wells 78-81. Four wells were infected with vector from a vector production in which vinblastine was added, wells 82-85. The values on the table are the percentage of positive cells on the plates, as measured by flow cytometry each time a plate was passaged. Shown at the top bar, 5,9,11, etc. are the number of days that have transpired between infection at day zero and passage and cytometry measurements in that column. For example, the first passage of V78 was 5 days post infection, the second passage of plate V78 was 11 days post infection, i.e. 6 days after the first passage; the first passage of V79 was 9 days post infection, the second passage of V79 was 16 days post infection: etc. The lower part of the panel, which is in bold typeface, is the same data as shown in the top part. In this lower part the data of the top part of the panel is normalized for easier comparisons. For example V78 is, at its first passage, set to 1.0. All the values for the V78-V81 plates are set relative to this value. For the V82-V85 plates, i.e. the infections with vinblastine-made vector, the numbers are relative to setting 1.0 for V82 at first passage.

This panel demonstrates two immediate results. First the percentage of GFP positive cells falls rapidly with each successive passage of the cells infected with non-vinblastine made vector, V78-V81, e.g. for V78 it is 2.11, 0.060, 0.025. When the data is normalized as shown in the lower part of the panel it is 1.0, 0.028, 0.012. This rapid decline is also seen in the plates passaged the first time at 9 days or 16 days, or 23 days post infection. The relative reductions with each passage in the plates infected with vinblastine-made vector, while substantial, is relatively much less. Compare for example 0.028 to 0.193 for V78 versus V82 in the normalized section of the panel. This relative reduction in successive passages of a plate is less for cells infected with the vinblastine-made vector whether first passage is at 5, 9, 16, or 23 days post infection.

The second observation is that in comparing the number of GFP expressing cells, at each first passage, there is, in the non-vinblastine vector infected plates V78-V81, a marked decline as the interval between infection and first passage increases: 2.11, 0.97, 0.71, 0.42. This is expected since Hep G2 cells even at confluence maintain a low but steady rate of cell division if they are well fed. In contrast the cells infected with vinblastine made vector do not show this decline when comparing later first harvests with earlier first harvests; 13.23, 12.10, 14.45, 13.96. These results can be explained by the loss or dilution of AAV-TRUF11 genomes due to the cell division that occurs in both sets of plates, being partially compensated for in plates V82-V85 by a continual resupply of AAV-TRUF11 genomes in these plates. (After infection, the vector particles and Episomes, which is the form that AAV-TRUF11 genomes have in the cell once the vector has uncoated, do not replicate. Consequently, the percentage of positive cells is continually diluted with cell division. Additionally, episomes are lost by degradation at cell division since most of them will remain outside the nuclear membrane when it re-forms after telophase.) This lack of decrease on the plates infected with vinblastine made vector is consistent with a much higher level of late uncoating in the vinblastine vector infected plates in comparison with the plates infected with the vector produced without vinblastine. When cells are passaged the media is first aspirated, then plates are washed in PBS. This is followed by a second wash with PBS which is then also aspirated. Only then are cells trypsinized. Media is added to the plates after trypsinization to render the trypsin non-functional. Cells are resuspended in this media and aliquots are passaged to a fresh plate. These several exchanges of media and PBS ensure that if any vector remains free in the media on the original plate it will be discarded at first passage.

AAV-TRUF11 genomes that have integrated into the host cell's genome will be replicated at each cell division and consequently not be lost at each 15:1 split. However, the lack of decrease in first passage measurements for V82 to V85 cannot be explained by the AAV-TRUF11 genomes having been integrated into the infected cell's genome because if that were so then the percentage of GFP expressing cells, which had fallen relatively less on these plates between the first and second passage would not subsequently show much additional decrease. However these values that had not fallen so much between the first and second passage subsequently fall greatly, especially between the second and third passage, e.g. 0.193 to 0.063, 0.330 to 0.071, 0.422 to 0.079, 0.403 to 0.082.

To illustrate the results of table 3, the following two figures from several of the above harvests are shown. FIG. 7 shows a dot plot of the flow cytometry of V78 and V82 at day 5 and day 11, i.e. the first and second harvest of each of these plates. Plate V82, the plate infected with vinblastine made virus has more GFP expressing cells than does Plate V78, the plate infected with non-vinblastine made virus. The relative difference between the two plates increases between the first and second passages. FIG. 8 shows a comparison of six plates from the day 23 harvest. V79 and V83 are from their 3^{rd} harvest. V80 and V84 are from their second harvest. V81 and V85 are from their first harvest. In all cases the plates infected with vinblastine made virus, V83, V84, and V85 have more GFP expressing cells than do the matching plates infected with the non-vinblastine made virus, V79, V80, and V81.

To emphasize the above findings, Table 4 is a comparison of the fold reduction in GFP expressing cells between the first and second harvests of infections V78-V85 illustrating the difference between the two vectors with respect to yet uncoated vectors.

The data of tables 3 and 4 demonstrates that on average the vector made with vinblastine transduces the infected cells more slowly than does the vector made without vinblastine. Possible mechanisms for this slower transduction, but not the only possibilities, might be slower migration to the nucleus, slower entrance into the nucleus, slower dissolution of the nucleocapsid enclosing the rAAV genome.

Table 5. Shows a comparison of infections using two additional vectors prepared in parallel, one without and one with vinblastine. The V86 and V87 infections were with vector made without vinblastine; The V88 and V89 plates were infected with vector made with vinblastine. As seen the difference at first passage between the two plates infected with vector from these two vector preparations is less substantial than the differences seen in the assay of Table 3. Compare 2.12 and 3.58. However, in comparing the first harvests at the 19^{th} day after infection there is a much more substantial difference. While the first harvest of V87 is less that the first harvest of V86 (0.970 vs 2.12) the first harvest of V89 is higher than the first harvest of V88 (5.090 vs 3.58). This is consistent with the uncoating in the vector used in V88-V89 occurring later than the vector used in plates V82-V85. This delayed uncoating is more than compensating for the dilution of Hep G2 cells due to the continuing division of Hep G2 cells in culture and consequent loss of episomes as explained above. This can explain why the vector used in V88-V89 seems initially to have been produced in relatively lower amounts than the vector used in plates V82-V85.

A conclusion from these results is not only that the vinblastine made vector uncoats more slowly but that the total number of transducing units in the vinblastine made production is greater than the 4-fold increase determined by measuring the number of transformed cells shortly after infection. By measuring only what is observed at first passage we are missing the vector that uncoats later. In addition, since the later uncoating vector is being diluted but these vector particles are not being replicated with cell division there is a further failure to measure total vector. For example. If 100 cells are infected with 10 vector particles and each vector particle is in a separate cell and all uncoat and express immediately the resulting measurement would be 10% implying 10 vector particles. If those 10 vector particles did not uncoat until after one cell division, there would now be 200 cells and a measurement, therefore, of only 5% if the same percentage of cells is measured at each assay. And so on with each cell division. (There are approximately 4 cell division between each passage.)

Table 6 shows 4 parallel infections in which HepG2 cells were grown to confluence on Matrigel coated plates, which restricts growth of HepG2 cells that have, as mentioned, a tendency to overgrow each other even when confluent. These are the same two vector preparations used in the assay of Table 3. The results are the same as shown previously on normal cell culture plates. There are more positive cells on the plate infected with the vinblastine made vector and the decrease in percent of positive cells with each 15:1 split is lower for this plate. A second comparison is shown in the second set. These cells were infected at the same time as the first set, but their first harvest was at 2 weeks postinfection rather than one week. Consistent with the previous results, the harvest of the vinblastine-vector infected plates is higher when harvested for the first time at two weeks post infection rather than one week post infection. The rise from 4.34 to 7.06 when comparing the first harvests of the two plates infected with vinblastine made vector is consistent with a lower cell turnover on Matrigel plates. The difference between the percentage of positive cells on the plates infected with vinblastine made vector and non-vinblastine made vector is approximately 4-fold on the plates harvested for the first time at one week while it has risen to approximately 14-fold on the plates harvested for the first time at two weeks.

The third data set is from a separate infection with the same two vector preparations. In this infection the first harvest is only 2 days post infection. This is the earliest point at which cells could be visibly seen to fluoresce green under UV light. The result is consistent with the vinblastine made vector not uncoating as quickly as the non-vinblastine made vector. A consideration might have been that with the non-vinblastine made vector, perhaps the Hep G2 cells were still dividing so quickly after reaching confluence that episomes were already lost at first passage and so there was an undercount. This data in which cellular replication is hindered by confluence and by plating on matrigel and in which, in this last comparison, harvesting is as soon after infection as green fluorescing cells can be detected does not support such a scenario.

### Table 7

An observation is that after infection, especially at later times after infection, many cells on plates infected with the vinblastine-made vector seem brighter than cells infected with vector made without vinblastine. (See previous flow cytometry figures) This might indicate that some cells infected with the vinblastine-made vector have been infected with more than one vector particle or that some of the vector genomes may have integrated into the AAVS1 site.

The wt AAV has an alternative infection pathway to the one described above in the Background section. The protein produced from it's Rep gene (designated as the Rep protein) can catalyze the integration of the wild type AAV genome into the host cell's genome through a mechanism that involves nicking at a site in chromosome 19, designated as the AAVS1 site. In standard gene therapy with rAAV vectors there is no Rep gene and therefore no Rep-dependent integration. Rep-dependent integration of rAAV genomes, however, can be achieved by a coinfection of the rAAV with wt AAV, which will supply the Rep protein in trans. The capacity of an rAAV vector genome to undergo Rep-dependent AAVS1 site specific integration can serve as a marker for a well-made rAAV virus.

Cells with AAV-TRUF11 genomes integrated into the AAVS1 site fluoresce, on average, more strongly than cells with episomal AAV-TRUF11 genomes or genomic integrations elsewhere than AAVS1. Cells with more than one non-integrated transducing vector would also give a stronger fluorescent signal. To determine more quantitatively whether there were disproportionately more bright cells in the plates infected with Vinblastine-made vector, the following assay was performed. 6 plates of confluent Hep G2 cells were infected with vector made either without vinblastine, V1, V2, V5 or with vinblastine, V3, V4, V6. V1-V4 were coinfected with wt AAV; V5 and V6 were not. In comparing the first passages of V1 and V3 by flow cytometry, V3 has 7× as many positive cells as does V1. Measurements of the number of cells in the bins with higher FITC levels, indicative of higher GFP expression, are shown. Bin 1 is the highest FITC level; Bin 2 is next highest etc. The higher expressing bins have a disproportionately high percentage of the positive cells. That is, the relative number of cells in the higher bins is more than 7-fold greater in the plates infected with vinblastine-made vector than in the plates infected with non-vinblastine-made vector. If the infected cells were infected with particles that infected randomly then the two populations should have had approximately the same 7-fold difference in each of the bins. (Since the plate infected with vinblastine-made virus has 7-fold more positive cells than the plate infected with non-vinblastine-made virus, the chances of multiple infections of one cell, even with random infections, will be higher. However, since the number of positive cells is still only 14%, the percentage of cells with multiple infections would still be low.) The deviation from this result means that either a subset of the cells infected with vinblastine-made virus are being infected with more than one vector particle in a manner that is not random or that a subset of the cells infected with the vinblastine made virus are producing higher levels of GFP per vector. The latter possibility is consistent with vector integration at AAVS1. However, the significant drop in percentage of GFP expressing cells with passaging is not consistent with a large fraction of the higher GFP expressing cells having integrated genomes. (See data of FIGS. 11 and 12 for a further examination of this point.) In comparing the first passages of plates V2 and V4, the results are similar except that at first passage there is a 15-fold difference between the plates with respect to the number of positive cells. Again, the plate that had been infected with the vinblastine made vector has higher numbers of brighter cells, i.e. more than 15-fold, which as with the V1, V3 comparison indicates a greater percentage of cells infected by more than one AAV-TRUF11 vector particle in the infections with vinblastine-made virus.

FIG. 12 explicitly demonstrate that with the infection of the vinblastine-made virus the infection of cells with more than one vector occurs at a higher frequency than would be expected if infection was random. In FIG. 12 the infection with vector #12 (i.e. a vinblastine-made vector) without added wt AAV is about 5% of the cells. Almost 40% of the 5% of GFP expressing cells, as approximately determined by a visual examination of the flow cytometry graph, have more than one expressing vector. With only 5% of the cells having vector, then the percentage having more than one vector would be negligible if successful vector transductions were, on a cell-by-cell basis, random and mutually independent. There must be some mechanism that renders cells which have one expressing vector more likely to acquire additional expressing vectors. An additional point made by FIG. 12 is that it is likely that the cells with more than one expressing vector have on average more than two such vectors per cell since the higher expressing peak is about 9-fold brighter than the lower expressing peak. The lower expressing peak is at the same expression level as the vector #7 infected cells, i.e. cells infected with non-vinblastine made virus. This phenomenon of cells transduced with multiple vectors did not occur with vectors made without vinblastine.

These results imply that measuring the number of cells that are positive for GFP expression after infection seriously underestimates the extent to which the vinblastine made vector has more transduction capable vector particles than does the non-vinblastine made vector.

An additional point relevant to this data: The presence of more than one rAAV genome in a cell is useful for treatments in which populations of cells are infected with more than one type of vector simultaneously, e.g. two vectors each containing one unique transgene, with the goal of producing some cells transformed by both vectors together. A more significant reason for having two vectors of different sequence in the same cell is to create the opportunity for recombination between two vector genomes when the gene of interest is too large to fit into one vector particle and must be divided into two parts.

The above results are from plates infected with vector from the cell pellet. In assays in which infection was with vector derived from media the difference between the number of higher FITC expressing cells was larger than the difference observed in plates infected with vector extracted from the cell pellets. This is shown in Table 8. (Table 8 shows the result in which 3-fold higher percentage of the total media preparation was used than the pellet preparation. Therefore, the number of cells in the media fraction could be expected to be ‘6’. Rather than ‘2’ for the plate infected with vector made without vinblastine. Table 8 shows that brighter cells, consistent with the presence of more than one uncoated vector particle, are more common in cells infected with vinblastine made vector than in cells with non-vinblastine made vector when the vector is from the media fraction. In this assay even with the 3-fold correction, the difference between the two plates remains substantial.

The decline in positive cells in V3 (Table 7) between the day 24 passage and the day 31 passage is only from 0.81 to 0.50. This is a relatively smaller drop than seen from day 10 to day 17. This suggests that the 0.50 value represents, at least in part, not more uncoating, but rather integration of the AAV-TRUF11 genome into the host cell's genome and consequently the persistence of the now integrated AAV-TRUF11 genome through cell division. The 0.50 and 0.80 values on plates V3 and V4 as well as the 0.83 value on plate V6 represent the highest percentage of cells that might still be GFP expressing due to their AAV-TRUF11 genomes being integrated. This is a value much higher than seen for cells infected with the non-vinblastine made vector but still represents only a small fraction of the total number of cells that expressed the GFP gene in plates V3,V4,V6.

A comparison of V1 and V3. With V5 and V6 shows that the presence of wt AAV did not affect the number of transducing units.

Paclitaxel, (CAS Number 33069-62-4) another microtubule inhibitor, (thought to work by a different mechanism than vinblastine) was also tested for a possible effect on vector production. Paclitaxel also gave more functional vector when included in vector production.

There is a large literature on both vinblastine and paclitaxel due to their usefulness in cancer therapy. This literature has revealed many effects on microtubule dynamics, including an inhibition of microtubule disassembly by paclitaxel, but also other less extensively characterized effects for both these agents, which effects may play a role in vector production.

Paclitaxel, like vinblastine, was added to plates 90 minutes prior to transfection. Paclitaxel was added to a concentration of 275 nM.

Table 9 shows flow cytometry data comparing the percentage of GFP expressing HepG2 cells in two parallel infections of confluent cells. One plate was infected with vector made with nothing added, i.e. #7; a second plate was infected with vector made with paclitaxel added, i.e. #19. In both plates, infection was performed with vector extracted from the cell pellet. Shown are the flow cytometry measurements at both the first and second passage. As seen at passage 1, paclitaxel production gives more transduction. There is a large decrease in the percentage of transduced cells at passage 2 in virus #7, but much less decrease in vector #19, the infection with paclitaxel-made vector. Since the cells were confluent at infection the large decrease between passage 1 and passage 2 for vector in #7 is to be expected. The AAV viruses have little ability to integrate into the genome of non-replicating cells, so the vector genomes remain episomal and are lost at cell division. The paclitaxel vector does not show this large decrease. (The flow cytometries of the second passage of each infection are shown in FIG. 9.) This result demonstrates a later uncoating for some of the paclitaxel vector. The gap between the first passage and the second passage encompasses about 4 cell divisions. Vectors that uncoat during this time can integrate their genomes because the cells are dividing and a certain, albeit low, percentage of this vector will integrate. In cells in which the vector uncoats but in which the vector genome does not integrate the vector genomes will be lost. The exception is cells in which a vector does not uncoat until after the last cell division before passaging. In those cells the episome will not yet be lost before flow cytometry. In the non-paclitaxel made vector there is little evidence of uncoating after the cells are passaged. In the paclitaxel made vector the flow cytometry indicates either uncoating after the 4^{th} cell division or uncoating after any of the cell divisions followed by some level of integration of the rAAV genome. Most likely both phenomena are occurring.

Table 10 shows infections of non-confluent Hep G2 cells, rather than the usual confluent cells, with vector made with nothing added compared to vector made with vinblastine or paclitaxel. Shown for each virus production are infections of Hep G2 cells by vector found in the cell pellet and vector exported to the media. Vector production with vinblastine produces, as described above, a vector that gives more transduction than vector produced with nothing added. Reducing the amount of vinblastine reduces the vinblastine effect. In Table 9 lower amounts of paclitaxel had been used. Both the vinblastine and the paclitaxel result in relative increases in the proportion of vector exported to media as compared with vector remaining in the producer cells. In this example with higher paclitaxel there is no transducing vector detected in the cell pellet. While this may indicate that all the vector made in these cells is exported it is possible that higher levels of paclitaxel in the producing cell, by some unknown mechanism, traps completed vector particles so as to render them unextractable.

Table 11 is a comparison of flow cytometry measurements of two additional infections of Hep G2 cells with vector made with paclitaxel. In this case, however, while the vector used to infect plate 49 had been made with paclitaxel alone, the vector used in the infection of plate 50 had been made with the addition of paclitaxel and vinblastine together to the producer cells. There is by the flow cytometry measurement of Table 11 no apparent synergy between paclitaxel and vinblastine in the production of the rAAV. This suggests that both agents are causing the enhanced production of rAAV by the same mechanism. Since the two agents are thought to interfere with microtubule assembly and disassembly by different mechanisms and have quite different chemical structures, the implication is that it is interference with microtubule functioning that is principally responsible for the observed enhancement in rAAV vector production and not some alternative effects of these two agents.

Whether vector made with vinblastine or paclitaxel was efficient at site-specific integration, as first described by Kotin et. al., was tested. In these assays the AAV Rep protein was supplied in trans to the HepG2 cells by a coinfection of wt AAV virus simultaneously with AAV-TRUF11 vector. The results of one assay are shown in FIG. 10. It has been shown that when rAAV genomes are integrated site-specifically into the genome they express at the FITC level of the clusters seen in FIG. 10 (slightly below 10⁴ at this voltage). AAV-TRUF11 genomes that are not integrated or integrated in a non-Rep supported mechanism, i.e. integrated randomly, express predominantly at lower FITC levels. Thus, a cluster of cells at this FITC serves as a crude assay for site-specific integration. F147 is, for comparison, a flow cytometry of an assay done at an earlier time of a standard infection of Hep G2 plates with wt virus and AAV-TRUF11 that is known to have had a high level of site-specific integration. All three of the dot plots shown were performed many passages post infection.

FIG. 11 shows an assay in which the infections with the GFP expressing vectors were both accompanied by infection with a wt virus to catalyze site-specific integration. On the plate receiving vector made without vinblastine there is little sign of site-specific integration, i.e. no peak at about 10⁴. The cells on the other plate were infected with a vector made with vinblastine; on this plate there is a large population of cells whose expression level is consistent with site-specific integration. The flow cytometry on this assay was performed at first passage. (Note that the raw flow cytometry data of FIG. 10 is plotting FITC against SSC, while FIG. 11 is plotting FITC against PE.) The vector made with vinblastine is giving a higher proportion of higher GFP expressing cells upon infection. The question remains whether these higher expressing cells are due to vinblastine made virus being better at wt AAV rep-directed site-specific integration or the tendency of vinblastine made vector to deliver more than one vector to infected cells.

To separates the high expression due to infection with vector made with vinblastine from the high expression due to site-specific integration, the assay of FIG. 12 was performed. In this assay, once again the cells infected with the vector made without vinblastine, i.e. #7, demonstrate few cells with high expression, i.e. only a very small peak at 10⁴, and therefore shows few cells with either multiple vectors or site-specific integration even though coinfected with wt AAV. In both plates infected with vector made with vinblastine, i.e. the #12 plates, there are a substantial number of cells that express GFP at higher levels both with and without wt AAV coinfection. On the #12 plate coinfected with wt AAV there are a greater number of high expressing cells than on the #12 plate not coinfected with wt AAV, demonstrating site-specific integration in the cells infected with the vinblastine made vector. However, in comparing the heights of the peak, at approximately 10⁴, between #12 without wt AAV coinfection and #12 with wt AAV coinfection it is apparent that the greater proportion of this peak can be accounted for by the vector having been vinblastine made rather than site-specifically integrated. The addition of wt AAV coinfection accounts for a lesser, albeit still substantial, component of this peak.

Two transfection methods, other than the CaCl₂ method described above, are sometimes employed with triple transfection protocols to drive plasmids into cells. The effect on Hep G2 transduction when vinblastine had been added to vector productions using these two other protocols was tested.

An alternative to transfection with CaCl₂ is “PEI transfection”

PEI is Polyethylenimine 25 kD linear from Polysciences (cat #23966-2). stock is at 1 ug/ul. In water.

replate HEK293T cells in DMEM. Leave o/n

next day have a plate about 60% confl.

Mix 3 plasmids into 200 ul serum free DMEM. (use same amount of each plasmid as with triple transfection method above.)

Add to this tube an amount of PEI equivalent to 3 times the mass of the 3 plasmids combined.

(For example it there was 3 ug of plasmid, add 9 ug of PEI.)

Incubate 15 minutes at room temperature.

Add DNA/PEI to plate of cells

In 2-3 hrs remove media and replace with DMEM with 10% FBS

Add vinblastine at this point at the same concentration as with the CaCl₂ transfection.

Harvest in 48-72 hrs.

Process the same as with the CaCl₂ method described above.

A second alternative is “lipofectamine transfection”. (lipofectamine is purchased from Thermo-Fisher and used as is.

Use plasmids at same concentration as in ‘triple transfection method.

Set up HEK293T cells for overnight culture as with the previous protocols.

Following day replace media on cells with serum free media

Add plasmids to 200 ul serum free DMEM.

Mix lipofectamine stock gently. Put 8 ul of this stock into a second tube of 200 ul DMEM without serum.

5′ room temp.

Combine DNA mix and lipofectamine mix. Approximately 200 ul each.

Mix gently. at least 20 minutes room temp incubation. May become cloudy.

Add the 400 ul to plated cells in well. Rock plate back and forth.

Put in incubator for 4-6 hrs

After 4-6 hrs replace media with DMEM containing serum and antibiotics.

add vinblastine-dilution.

(In FIG. 13, addition of vinblastine before transfection is compared with addition after the post transfection media change, with the latter giving a superior result. It is likely that giving vinblastine at both times might be optimal.)

Harvest 48-72 hrs.

Harvest of plates and processing in all three methods is the same and is as described with the CaCl₂ method above

In each of the three methods, final concentration of Vinblastine is 100 nM.

The final concentration of Paclitaxel is 275 nM.

As seen in Table 12 with the PEI protocol and in FIG. 13 with the lipofectamine protocol the addition of vinblastine gives vector that demonstrates increased transduction in both cases. In the lipofectamine case while the final level of GFP expressing cells is no greater for vector extracted from the media than with vector from the cell pellet it does seem that the percentage of vector in the media compared with the vector in the cell pellet increases with vinblastine addition. In the case of lipofectamine the difference between the amount of transduction with vector made with vinblastine addition after media change during vector production is 10-fold greater than the transduction in cells infected with vector made without any vinblastine. It is likely that the better result with vinblastine added to producer cells after the 4-6 hr media change as compared with addition before lipofectamine and plasmid addition is that in the former case vinblastine was present during a much longer fraction of the viral production. An alternative possibility is that transfection may work best in some cases if it occurs before the disrupting agent is added to the producer cells.

These last two assays are consistent with the notion that disruption of normal intracellular transport and communications is likely to improve production in any cells where proteins produced from separate genetic entities are required to cooperate in vector production.

However, the disruptions to intracellular processes seen with vinblastine and paclitaxel are likely to contribute to an enhanced vector production by additional mechanisms. The enhancement to vector production found in the assays of this application was, based on the results of van Lieshout et. al. and Yang et. al., larger than expected, At present an innate immune response to the components of vector assembly and a failure of most cells in a producer population to be assembling complete viral particles have been identified as significant roadblocks in vector production. These phenomena, as well as other presently unknown issues, may also be affected by the microtubule inhibitors.

This method produces rAAV that provides higher transduction levels than does the standard method. The increased transduction capacity of the vector made by the modification herein described is not only an increase in the number of transduced cells at the initial infection but also encompasses a later uncoating of vector that is not counted when counting the transduced cells soon after the initial infection. It also encompasses a marked increase in the percentage of cells with more than one uncoated vector. Again, the additional vectors per cell are not counted if one counts only how many cells are expressing GFP. The number of cells with more than one vector is higher than if transduction was random as apparently is the case with the vector made the standard way. This indicates that that the vinblastine-made vectors identify a subset of the target cell population predisposed to multiple transductions. These vectors must enter the cell through a canonical pathway rather than being taken up by the cell non-specifically by many different mechanisms. This is consistent with the vinblastine made vector being a better vector that one made without vinblastine. It has been demonstrated using an in vitro vector assembly system that poorly assembled vector particles can be taken up by cultured cells and that the genome within these particles can be expressed.

The tendency to transduce cells with more than one vector can be very useful. AAV is a small virus and so successful transduction with larger genes requires that the gene be split into separate particles and that the genes released from those particles recombine in the infected cells.

rAAV vector made with this modification transduce target cells more slowly, than vector produced by the standard method. This slower transduction may relieve some of the immunological stress that is believed to be, at least in part, due to a sudden surge to the cell surface of capsid peptides from the now dissociated viral-vector particles.

Vector produced with this modification has several additional differences.

Vector particles produced by this method are more likely to be exported from the producer cell into the surrounding culture media than vector produced by the standard method.

Export to the media seems to correlate with a better made virus. Vector exported from the cell is easier to purify and results in cleaner preparations since most cellular proteins and nucleic acids are retained by the cell. It is widely believed, though unreported, that some of the negative effects of rAAV treatment on patients are due to cellular contaminants in vector preparations.

(Note: The AAV serotype employed in the assays of this application was AAV2. Vectors made from other serotypes have been found to give greater amounts of vector exported to the media with standard rAAV production than vector made with AAV2.)

The vinblastine-made rAAV vector is efficient at integrating into the host cell's genome in a AAV Rep protein dependent manner. An ability to undergo Rep-dependent integration may be used in future gene therapy approaches.

In addition, the ability to participate in Rep-dependent integration is likely a marker for a better made vector. In standard vector production the small amount of virus exported to the media is substantially better at Rep-protein-supported integration than virus remaining in the cell pellet. Vector exported from the cell is more likely to be vector that has completed all the steps in viral assembly. In vector made with vinblastine, however, even vector remaining in the cell pellet is efficient at Rep-supported genomic integration. This is a marked difference between vinblastine preparations and non-vinblastine preparations.

In conclusion this method gives more vector and more transduction from this vector. There are also four functional indications that vector so produced is a better vector than vector produced by standard methods: greater export of vector from producer cells into media, enhanced site-specific integration when supported by the Rep protein in trans, transductions of cells by multiple vectors, and a longer time between infection of cells and expression of the transgene.

There are other approaches to producing rAAV vectors than the transfection of 3 or fewer plasmids, one of which requires more than 3 plasmids and one of which involves less or no transfected plasmids.

The first approach:

The Rep and Cap genes are normally considered as only two genes since they are two open reading frames. However, the Rep gene encodes 4 proteins dependent on different start, stop, and splicing sites. The Cap gene encompasses 3 proteins depending on 3 start sites as well as an assembly factor coded by a short internal sequence in an alternate reading frame. Numerous studies have shown that the ratios of the proteins have significant effects on the efficiency of vector production. With the separation of parts of the Rep Cap sequences into different plasmids with different control elements it is possible to increase vector yield. While this increases the number of plasmids the control over the relative amounts of proteins produced from each gene seems to compensate for this. It is likely that this approach may, in the future, provide a viable method for increased vector yields and the method of this application should complement and be compatible with this multi plasmid approach.

The second approach:

This method is to construct cell lines that have the necessary genes for viral production embedded in the cell's chromosomes. At present many technical problems remain with this approach, but if those problems can be solved the protocol described in this application may increase yields of vector produced in cell lines. It may do so either by means of less directed protein migrations, thereby causing increased protein proximity or, as in the transfection protocol, by as yet unknown mechanisms.

A major effort is also being made to develop AAV capsid variants for directed targeting of vector to specific cell types. The list of capsid variants presently in pre-clinical trials is expanding; improved production methods, to be optimally useful, should be able to accommodate newly developed capsid variants with little adjustment to the production methods. The modification of this application meets that requirement.

The method of this application since it involves the addition of soluble agents to the vector production reactor is easily incorporated into established production protocols. The method is scalable. The method does not require plasmids that are hard to construct and difficult to produce in large quantities such as those that are large or have the Rep gene and the AAV ITRs together on one plasmid.

Combining this method with other protocols for improving product yield such as those of van Lieshout et. al., Yang et. al., and Emmerling et. al may provide synergistic advantages.

This invention is not limited to the particular embodiment described herein. The terminology herein is intended to describe only this particular embodiments only, and is not intended to be limiting. One skilled in the art will know that each academic laboratory and commercial production facility uses modifications and variations on the above procedures and those skilled in the art can adapt the methods of this patent to their circumstances. In particular the concentrations of disrupting agents and the timing of their addition to the producer cells may be quite different in different production facilities and will need to be adjusted in each facility for optimal results. This invention can, in addition, be employed with yet to be developed methods for vector production that use multiple plasmids in producer cells or where it is found that disruption to intracellular trafficking and communications is advantageous. In addition, it can be used with other cell lines e.g. Sf9 which is already in use in rAAV vector production or CHO, or yeast cells line which are suggested alternatives to HEK293 and HEK293T. Those skilled in the art will readily think of other agents and other methods for disturbing internal communications and transport in the cell as well as other cells and types of vectors whose production might be enhanced.

It is also understood that the patent does not depend on the correctness of the theoretical aspects presented and is not to be bound by them.

### REFERENCES

1. J. S. Bartlett, R. Wilcher, R. J. Samulski, Infectious entry pathway of adeno-associated virus and adeno-associated virus vectors. J Virol 74, 2777-2785 (2000).
2. B. L. Ellis et al., A survey of ex vivo/in vitro transduction efficiency of mammalian primary cells and cell lines with Nine natural adeno-associated virus (AAV1-9) and one engineered adeno-associated virus serotype. Virol J 10, 74 (2013).
3. N. Zeltner, E. Kohlbrenner, N. Clément, T. Weber, R. M. Linden, Near-perfect infectivity of wild-type AAV as benchmark for infectivity of recombinant AAV vectors. Gene Ther 17, 872-879 (2010).
4. J. J. Aponte-Ubillus et al., Molecular design for recombinant adeno-associated virus (rAAV) vector production. Appl Microbiol Biotechnol 102, 1045-1054 (2018).
5. L. T. Collins, S. Ponnazhagan, D. T. Curiel, Synthetic Biology Design as a Paradigm Shift toward Manufacturing Affordable Adeno-Associated Virus Gene Therapies. ACS Synth Biol 12, 17-26 (2023).
6. D. Duan, Lethal immunotoxicity in high-dose systemic AAV therapy. Mol Ther 31, 3123-3126 (2023).
7. A. Kruzik et al., Prevalence of Anti-Adeno-Associated Virus Immune Responses in International Cohorts of Healthy Donors. Mol Ther Methods Clin Dev 14, 126-133 (2019).
8. P. Ward, N. Clément, R. M. Linden, cis effects in adeno-associated virus type 2 replication. J Virol 81, 9976-9989 (2007).
9. L. P. van Lieshout et al., A novel dual-plasmid platform provides scalable transfection yielding improved productivity and packaging across multiple AAV serotypes and genomes. Mol Ther Methods Clin Dev 29, 426-436 (2023).
10. R. Yang et al., AAVone: A Cost-Effective, Single-Plasmid Solution for Efficient AAV Production with Reduced DNA Impurities. bioRxiv, 2025.2001.2007.631712 (2025).
11. X. Xiao, J. Li, R. J. Samulski, Production of high-titer recombinant adeno-associated virus vectors in the absence of helper adenovirus. J Virol 72, 2224-2232 (1998).
12. M. Nonnenmacher, H. van Bakel, R. J. Hajjar, T. Weber, High capsid-genome correlation facilitates creation of AAV libraries for directed evolution. Mol Ther 23, 675-682 (2015).
13. S. Zolotukhin, M. Potter, W. W. Hauswirth, J. Guy, N. Muzyczka, A “humanized” green fluorescent protein cDNA adapted for high-level expression in mammalian cells. J Virol 70, 4646-4654 (1996).
14. D. Grimm, M. A. Kay, J. A. Kleinschmidt, Helper virus-free, optically controllable, and two-plasmid-based production of adeno-associated virus vectors of serotypes 1 to 6. Mol Ther 7, 839-850 (2003).
15. C. A. Laughlin, J. D. Tratschin, H. Coon, B. J. Carter, Cloning of infectious adeno-associated virus genomes in bacterial plasmids. Gene 23, 65-73 (1983).
16. P. Ward, C. E. Walsh, Targeted integration of a rAAV vector into the AAVS1 region. Virology 433, 356-366 (2012).
17. R. M. Kotin et al., Site-specific integration by adeno-associated virus. Proc Natl Acad Sci USA 87, 2211-2215 (1990).
18. C. Zhang, N. G. Cortez, K. I. Berns, Characterization of a bipartite recombinant adeno-associated viral vector for site-specific integration. Hum Gene Ther 18, 787-797 (2007).
19. H. Nakai, T. A. Storm, M. A. Kay, Increasing the size of rAAV-mediated expression cassettes in vivo by intermolecular joining of two complementary vectors. Nat Biotechnol 18, 527-532 (2000).
20. S. Dash, D. M. Sharon, A. Mullick, A. A. Kamen, Only a small fraction of cells produce assembled capsids during transfection-based manufacturing of adeno-associated virus vectors. Biotechnol Bioeng 119, 1685-1690 (2022).
21. C. H. Chung et al., Production of rAAV by plasmid transfection induces antiviral and inflammatory responses in suspension HEK293 cells. Mol Ther Methods Clin Dev 28, 272-283 (2023).
22. X. Zhou, N. Muzyczka, In vitro packaging of adeno-associated virus DNA. J Virol 72, 3241-3247 (1998).
23. V. V. Emmerling et al., Rational plasmid design and bioprocess optimization to enhance recombinant adeno-associated virus (AAV) productivity in mammalian cells. Biotechnol J 11, 290-297 (2016).
24. J. Becker, J. Fakhiri, D. Grimm, Fantastic AAV Gene Therapy Vectors and How to Find Them-Random Diversification, Rational Design and Machine Learning. Pathogens 11, (2022).

1. A modification to standard methods of producing gene therapy vectors to produce vectors that provide enhanced transduction which method comprises disrupting cellular trafficking and communications networks.

2. The method of claim 1 in which directed trafficking of molecules within the cell is disrupted.

3. The method of claim 1 in which microtubule functioning is disrupted.

4. The method of claim 1 in which trafficking and communications networks are disrupted by vinblastine.

5. The method of claim 1 in which trafficking and communications networks are disrupted by paclitaxel.

6. The method of claim 1 in which more vector particles are produced.

7. The method of claim 1 in which vector particles are produced that are more efficient transduction agents.

8. The method of claim 1 in which vector particles are produced that transduce infected cells more gradually.

9. The method of claim 1 in which vector particles are produced that have a proclivity to transduce cells with more than one functional vector per transduced cell.

10. The method of claim 1 in which vector particles are produced that have a enhanced ability to integrate their genomes in an AAV Rep dependent manner if the Rep protein is supplied in these cells.

11. The method of claim 1 in which vector particles are existing adeno-associated virus and recombinant adeno-associated virus as well as those that will be developed in the future including the known AAV serotypes: AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9(AAV-9), AAV type 10 (AAV-10), AAV type 11 (AAV-11), as well as modified AAVs and non human AAVs; AAVbb2, AAVcy5, AAVrh10, AAVrh20, AAVrh39, AAVrh43,AAVrh64R1, AAVhu37avian AAV, bovine AAV, canine AAV, equine AAV, primate AAV, non-primate AAV, ovine AAV, as well as AAVs comprising a capsid protein of one AAV subtype and genomic material of another subtype, and AAVs with mutant or chemically modified capsid protein, or AAVs in which the capsid is chimeric i.e. an AAV capsid with regions derived from more than one AAV serotype or other parvoviruses, and the related, but non-AAV parvoviruses or sequences from these parvoviruses, e.g. bocaviruses, goose parvovirus, and others.

12. The method of claim 1 in which cells for vector production are HEK293 cells and derivatives and modifications of HEK293 cells.

13. The method of claim 1 in which produced vector is used to infect cells that are dividing or that are soon likely to divide.

14. The method of claim 1 in which genetic elements required for vector production are introduced into the producer cells by transfection.
